(19) United States
(12) Patent Application Publication
(10) Pub. No.: US 2005/0120409 A1
(43) Pub. Date: Jun. 2, 2005

(54) Pseudomonas syringae harpins, hopptop and hoppmahpto, and their uses

(76) Inventors: Alan Collmer, Ithaca, NY (US); Adela Ramos, Silver Spring, MD (US)

Correspondence Address: Michael L Goldman;Nixon Peabody, Clinton Square, P O Box 31051, Rochester, NY, 14603-1051, US

(21) Appl. No.: 10/504,048

(22) PCT Filed: Jan. 30, 2003
(86) PCT No.: PCT/US03/03165

Related U.S. Application Data
(60) Provisional application No. 60/356,408, filed on Feb. 12, 2002.
(60) Provisional application No. 60/380,185, filed on May 10, 2002.

(51) Int. Cl. A01H 1/00; C12N 15/82; C12N 15/87; C07H 21/04; C12N 9/24

(52) U.S. Cl. 800/279; 800/287; 435/69.1; 435/468; 435/200; 435/419; 536/23.2

(57) ABSTRACT

The present invention is directed to isolated proteins or polypeptides which elicit a hypersensitive response in plants, as well as isolated DNA molecules which encode the hypersensitive response eliciting proteins or polypeptides. These isolated proteins or polypeptides and the isolated DNA molecules can be used to impart disease resistance, stress resistance, and enhanced growth to plants or plants grown from treated seeds, to control insects on plants or plants grown from treated plant seeds, to impart post-harvest disease or desiccation resistance in fruits or vegetables, to impart enhanced longevity of fruit or vegetable ripeness, to impart desiccation resistance to cuttings of ornamental plants, and/or promote early flowering of ornamental plants, either by topical application of the proteins or polypeptides or transgenic expression in recombinant plants or plant seeds.

This application is entitled to priority benefit of U.S. Provisional Patent Application Ser. No. 60/356,408, filed Feb. 12, 2002, and U.S. Provisional Patent Application Ser. No. 60/380,185, filed May 10, 2002, each of which is hereby incorporated by reference in its entirety.

This invention was developed with government funding under National Science Foundation Grant Nos. MCB 9631530, MCB-9982646, and DBI-0077622. The U.S. Government may retain certain rights.

### FIELD OF THE INVENTION

The present invention relates to new hypersensitive response elicitor proteins or polypeptides of Pseudomonas syringae and their uses.

### BACKGROUND OF THE INVENTION

Interactions between bacterial pathogens and their plant hosts generally fall into two categories: (1) compatible (pathogen-host), leading to intercellular bacterial growth, symptom development, and disease development in the host plant; and (2) incompatible (pathogen-nonhost), resulting in the hypersensitive response, a particular type of incompatible interaction occurring, without progressive disease symptoms. During compatible interactions on host plants, bacterial populations increase dramatically and progressive symptoms occur. During incompatible interactions, bacterial populations do not increase, and progressive symptoms do not occur.

The hypersensitive response (“HR”) is a rapid, localized necrosis that is associated with the active defense of plants against many pathogens (Horsfall et al., eds., Plant Disease: An Advanced Treatise, Vol. 5, pp. 201-224, New York, N.Y.: Academic Press (1980); Mount et al., eds., Phytopathogenic Prokaryotes, Vol. 2, pp. 149-177, New York, N.Y.: Academic Press (1982)). The hypersensitive response elicited by bacteria is readily observed as a tissue collapse if high concentrations (≧10⁷ cells/ml) of a limited host-range pathogen like Pseudomonas syringae or Erwinia amylovora are infiltrated into the leaves of nonhost plants (necrosis occurs only in isolated plant cells at lower levels of inoculum) (Klement, Nature 199:299-300 (1963); Klement et al., Phytopathology 54:474-477 (1963); Turner et al., Phytopathology 64:885-890 (1974); Mount et al., eds., Phytopathogenic Prokaryotes, Vol. 2., pp. 149-177, New York, N.Y.: Academic Press (1982)). The capacities to elicit the hypersensitive response in a nonhost and be pathogenic in a host appear linked. As noted by Mount et al., eds., Phytopathogenic Prokaryotes, Vol. 2., pp. 149-177, New York, N.Y.: Academic Press (1982), these pathogens also cause physiologically similar, albeit delayed, necroses in their interactions with compatible hosts. Furthermore, the ability to produce the hypersensitive response or pathogenesis is dependent on a common set of genes, denoted hrp (Lindgren et al., J. Bacteriol. 168:512-22 (1986); Willis et al., Mol. Plant-Microbe Interact. 4:132-138 (1991)). Consequently, the hypersensitive response may hold clues to both the nature of plant defense and the basis for bacterial pathogenicity.

The hrp genes are widespread in gram-negative plant pathogens, where they are clustered, conserved, and in some cases interchangeable (Willis et al., Mol. Plant-Microbe Interact. 4:132-138 (1991); Dangl, ed., Current Topics in Microbiology and Immunology: Bacterial Pathogenesis of Plants and Animals—Molecular and Cellular Mechanisms, pp. 79-98, Berlin: Springer-Verlag (1994)). Several hrp genes encode components of a protein secretion pathway similar to one used by Yersinia, Shigella, and Salmonella spp. to secrete proteins essential in animal diseases (Van Gijsegem et al., Trends Microbiol. 1:175-180(1993)). In E. amylovora, P. syringae, and P. solanacearum, hrp genes have been shown to control the production and secretion protein elicitors of the hypersensitive response (He et al., Cell 73:1255-1266 (1993); Wei et al., J. Bacteriol. 175:7958-7967 (1993); Arlat et al., EMBO J. 13:543-553 (1994)). Hypersensitive response elicitor proteins, designated harpins, are proteins found in phytopathogens containing a type III secretion system and are typically glycine-rich, acidic, cysteine-lacking, heat stable proteins (He et al., Cell 73: 1255-1266 (1993).

The first of these proteins was discovered in E. amylovora Ea321, a bacterium that causes fire blight of rosaceous plants, and was designated harpin (Wei et al., Science 257:85-88 (1992)). Mutations in the encoding hrpN gene revealed that harpin is required for E. amylovora to elicit a hypersensitive response in nonhost tobacco leaves and incite disease symptoms in highly susceptible pear fruit. The P. solanacearum GMI1000 PopA1 protein has similar physical properties and also elicits the hypersensitive response in leaves of tobacco, which is not a host of that strain (Arlat et al., EMBO J. 13:543-53 (1994)). However, P. solanacearum popA mutants still elicit the hypersensitive response in tobacco and incite disease in tomato. Thus, the role of these glycine-rich hypersensitive response elicitors can vary widely among gram-negative plant pathogens.

Other plant pathogenic hypersensitive response elicitors have been isolated, cloned, and sequenced from various organisms, including: HrpW from Erwinia amylovora (Kim et al., J. Bacteriol. 180(19):5203-5210 (1998)); HrpN from Erwinia chrysanthemi (Bauer et al., MPMI 8(4): 484-91 (1995)); HrpN from Erwinia carotovora (Cui et al., MPMI 9(7): 565-73 (1996)); HrpN from Erwinia stewartii (Ahmad et al., 8th Int'l. Cong. Molec. Plant-Microb. Inter. Jul. 14-19, 1996 and Ahmad et al., Ann. Mtg. Am. Phytopath. Soc. Jul. 27-31, 1996); hreX from Xanthomonas campestris U.S. Patent Application Publ. No. 20020066122 to Wei et al.); HrpZ from Pseudomonas syringae pv. syringae (He et al., Cell 73:1255-1266 (1993); WO 94/26782 to Cornell Research Foundation, Inc.); and HrpW from Pseudomonas syringae pv. tomato (Charkowski et al., J. Bacteriol. 180:5211-5217 (1998)).

In electron microscopy studies, both HrpW and HrpZ of Pseudomonas syringae are associated with the type III secretion system pilus (Jin et al., Science 294:2556-2558 (2001); Jin et al., Molecular Microbiology 40:1129-1139 (2001)), suggesting that harpins work with the pilus to facilitate protein delivery into the plant cell. A P. syringae strain containing chromosomal deletions of hrpZ and hrpW has a reduced ability to cause the HR on nonhost plants, but it retains normal virulence on tomato (Charkowski et al., J. Bacteriol. 180:5211-5217 (1998)). This phenotype indicates that it is likely that there are more harpins in the genome.

The present invention is a further advance in the effort to identify, clone, and sequence hypersensitive response elicitor proteins or polypeptides from plant pathogens.

### SUMMARY OF THE INVENTION

The present invention is directed to isolated proteins or polypeptides which elicit a hypersensitive response in plants as well as isolated DNA molecules which encode the hypersensitive response eliciting proteins or polypeptides.

The hypersensitive response eliciting proteins or polypeptides can be used to impart disease resistance, stress resistance, and enhanced growth to plants or plants grown from treated seeds, to control insects on plants or plants grown from treated plant seeds, to impart post-harvest disease or desiccation resistance in fruits or vegetables, to impart enhanced longevity of fruit or vegetable ripeness, to impart desiccation resistance to cuttings of ornamental plants, and/or promote early flowering of ornamental plants. This involves applying the hypersensitive response elicitor protein or polypeptide in a non-infectious form to plants, plant seeds, cuttings removed from plants, and/or fruits or vegetables removed from plants under conditions effective to impart disease resistance, stress resistance, and enhanced growth to plants or plants grown from treated seeds, to control insects on plants or plants grown from treated plant seeds, to impart post-harvest disease or desiccation resistance in fruits or vegetables, to impart enhanced longevity of fruit or vegetable ripeness, to impart desiccation resistance to cuttings of ornamental plants, and/or promote early flowering of ornamental plants.

As an alternative to applying the hypersensitive response elicitor protein or polypeptide to plants or plant seeds in order to impart disease resistance, stress resistance, and enhanced growth to plants or plants grown from treated seeds, to control insects on plants or plants grown from treated plant seeds, to impart post-harvest disease or desiccation resistance in fruits or vegetables, to impart enhanced longevity of fruit or vegetable ripeness, to impart desiccation resistance to cuttings of ornamental plants, and/or promote early flowering of ornamental plants, transgenic plants or plant seeds can be utilized. When utilizing transgenic plants, this involves providing a transgenic plant transformed with a DNA molecule encoding a hypersensitive response elicitor protein or polypeptide and growing the plant under conditions effective to impart disease resistance, stress resistance, and enhanced growth to plants, to control insects on plants, to impart post-harvest disease or desiccation resistance in fruits or vegetables, to impart enhanced longevity of fruit or vegetable ripeness, to impart desiccation resistance to cuttings of transgenic ornamental plants, and/or promote early flowering of transgenic ornamental plants. Alternatively, a transgenic plant seed transformed with the DNA molecule encoding a hypersensitive response elicitor protein or polypeptide can be provided and planted in soil. A plant is then propagated under conditions effective to impart disease resistance, stress resistance, and enhanced growth to plants grown from the transgenic seeds, to control insects on plants grown from the transgenic plant seeds, to impart post-harvest disease or desiccation resistance in fruits or vegetables, to impart enhanced longevity of fruit or vegetable ripeness, to impart desiccation resistance to cuttings of the transgenic ornamental plants, and/or promote early flowering of the transgenic ornamental plants.

The present invention is also directed to a composition including a carrier and a protein or polypeptide of the present invention which elicits a hypersensitive response in plants. The composition may also include an additive such as fertilizer, insecticide, fungicide, nematacide, and mixtures of these additives.

### BRIEF DESCRIPTION OF THE DRAWINGS

FIG. 1 illustrates a comparison between the amino acid sequences of HrpZ, HrpW, HopPtoP, and HopPmaH from Pseudomonas syringae. Harpin proteins were analyzed using BLAST, and the percent similarities shown were determined using protein-protein BLAST. Like HrpZ and HrpW, two previously identified harpins (see He et al., Cell 73:1255-1266 (1993); Charkowski et al., J. Bacteriol. 180:5211-5217 (1998), each of which is hereby incorporated by reference in its entirety), HopPtoP and HopPmaH_{Pto} have N-terminal domains with no predicted function. The C-terminal domains of HopPtoP and HopPmaH_{Pto} show homologies to protein domains with enzymatic function. Lytic transglycosylases normally break down peptidoglycan in the bacterial cell wall, while pectin and pectate lyases break down pectin in the plant cell wall.

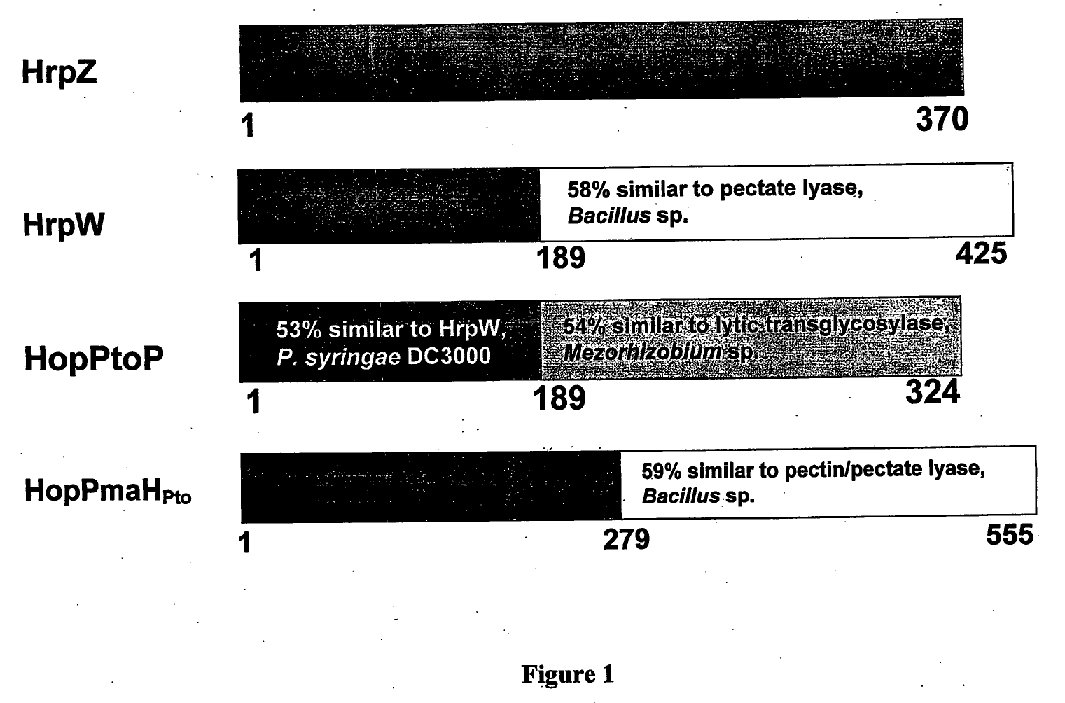

FIG. 2 shows the results of protein infiltration onto leaf surfaces and the resulting elicitation of a hypersensitive response (“HR”). HrpZ, HopPtoP, and HopPmaH_{Pto} were purified using a 6×His tag system, and denatured at 100° C. for 10 minutes. Proteins were infiltrated into Nicotiana tabacum cv. Xanthi, and the hypersensitive response was photographed at 24 hours.

FIGS. 3A-B illustrate the results of a secretion assay on HopPtoP and HopPmaH_{Pto}, respectively. HopPtoP and HopPmaH_{Pto} were tagged at the C-terminus with the adenylate cyclase (CyaA) protein for detection in subsequent Western analysis of the cell pellet (cell) and supernatant (SN). The plasmids were expressed in either P. syringae DC3000 or P. syringae DC3000 containing a non-functional type III secretion system (Δhrp/hrc). Both proteins are secreted by P. syringae DC3000 but not P. syringae DC3000 (Δhrp/hrc).

FIG. 4 is a graph depicting the results of a translocation assay on HopPtoP and HopPmaH_{Pto} relative to HrpW and HrpZ. Each harpin gene was fused with the adenylate cyclase (CyaA) reporter gene on an inducible plasmid, and expressed in either P. syringae DC3000 or P. syringae DC3000 containing a non-functional type III secretion system (Δhrp/hrc). CyaA is activated by the protein calmodulin, which is found only in eukaryotic cells, and produces cAMP from ATP. Increased cAMP levels can be measured, thereby detecting levels of translocation. The strains containing the CyaA fusions were infiltrated into tomato cv. Moneymaker plants, and leaf discs were collected 8 hours post-infiltration for analysis of cAMP levels. Both HopPtoP and HopPmaH_{Pto} are translocated into leaf tissues in the presence of a type III secretion system.

### DETAILED DESCRIPTION OF THE INVENTION

The present invention relates to an isolated DNA molecule having the nucleotide sequence of SEQ ID NO: 1 as follows:

| | | | | | | |
|---|---|---|---|---|---|---|
| atgaccatgg | gtgtttcacc | tattcgtaac | tcaaactccc | tgccgatcga | tttttcgtcg | 60 |
| ttgagcgcaa | agagtggcgg | gcataacggg | ctgggcagcg | gagacaattc | gactatcgac | 120 |
| ccgagtacgt | tgttgttcgg | caatcaaggg | cagacgcagg | tcaatttcgc | tccgcccaac | 180 |
| agcacggact | cctcaaccag | cggtgtgaac | gctgcgtcag | gcaatacggc | gtccggcctg | 240 |
| gtcgagcaaa | tcatgagcct | gctgaaacaa | ttgatgcaga | tgctgatgca | aaacaacaat | 300 |
| gcttccggta | accctcagac | tgattcgtca | acgccaggcg | tcggcagtgg | caacagcgtc | 360 |
| gggagcggcg | gtactggaag | cagtctcgca | ggcagtgacg | gtggcgacga | aacgtccggt | 420 |
| gtcggtaacg | gcggtttagg | cgacgcgggc | agcacgccaa | caacgagcgc | ggccgatggt | 480 |
| gtgccctcgg | atacttcact | cacgggtagc | ggtgggctgc | atttgcctca | acagcttgag | 540 |
| cagtatcgag | gcgacattat | ggacgccgcc | aaagccaccg | gcgtgccgcc | cagcgtgatc | 600 |
| gccgggcaga | tatgggctga | gtcgcgcggt | cagttgaatg | cggccaccac | caatgtcaac | 660 |
| ggcaaggccg | atgcgggcct | gatgcaggtc | aacgcagaca | cgttcaagtc | attgcagcag | 720 |
| caaaacccgg | ggttgctggg | caacgacgtc | aacgattcgc | acaccaacat | catggcgggc | 780 |
| gcgctctacc | tgcgagacca | gaacaaggag | ttcggcgaca | tgggggcagc | acttcgcgca | 840 |
| tacaactccg | ggcccgacaa | ggtcaataaa | gccgacctca | gcgacacggg | aggcgtgggc | 900 |
| ggcagcagct | acccggcgga | cgtactgaac | ttcgcgaaaa | tcatcgagag | tgggcagggc | 960 |
| aatttacccg | cttga | | | | | 975 |

This DNA molecule of the present invention encodes a protein or polypeptide having the amino acid sequence of SEQ ID NO: 2 as follows:

```
Met Thr Met Gly Val Ser Pro Ile Arg Asn Ser Asn Ser Leu Pro Ile
    1 5 10 25
Asp Phe Ser Ser Leu Ser Ala Lys Ser Gly Gly His Asn Gly Leu Gly
                          20 25 30
Ser Gly Asp Asn Ser Thr Ile Asp Pro Ser Thr Leu Leu Phe Gly Asn
                  35 40 45
Gln Gly Gln Thr Gln Val Asn Phe Ala Pro Pro Asn Ser Thr Asp Ser
          50 55 60
Ser Thr Ser Gly Val Asn Ala Ala Ser Gly Asn Thr Ala Ser Gly Leu
  65 70 75 80
Val Glu Gln Ile Met Ser Leu Leu Lys Gln Leu Met Gln Met Leu Met
                                  85 90 95
Gln Asn Asn Asn Ala Ser Gly Asn Pro Gln Thr Asp Ser Ser Thr Pro
                        100 105 110
Gly Val Gly Ser Gly Asn Ser Val Gly Ser Gly Gly Thr Gly Ser Ser
                115 120 125
Leu Ala Gly Ser Asp Gly Gly Asp Glu Thr Ser Gly Val Gly Asn Gly
        130 135 140
Gly Leu Gly Asp Ala Gly Ser Thr Pro Thr Thr Ser Ala Ala Asp Gly
145 150 155 160
Val Pro Ser Asp Thr Ser Leu Thr Gly Ser Gly Gly Leu His Leu Pro
                                165 170 175
Gln Gln Leu Glu Gln Tyr Arg Gly Asp Ile Met Asp Ala Ala Lys Ala
                        180 185 190
Thr Gly Val Pro Pro Ser Val Ile Ala Gly Gln Ile Trp Ala Glu Ser
                195 200 205
Arg Gly Gln Leu Asn Ala Ala Thr Thr Asn Val Asn Gly Lys Ala Asp
        210 215 220
Ala Gly Leu Met Gln Val Asn Ala Asp Thr Phe Lys Ser Leu Gln Gln
225 230 235 240
Gln Asn Pro Gly Leu Leu Gly Asn Asp Val Asn Asp Ser His Thr Asn
                                245 250 255
Ile Met Ala Gly Ala Leu Tyr Leu Arg Asp Gln Asn Lys Glu Phe Gly
                        260 265 270
Asp Met Gly Ala Ala Leu Arg Ala Tyr Asn Ser Gly Pro Asp Lys Val
                275 280 285
Asn Lys Ala Asp Leu Ser Asp Thr Gly Gly Val Gly Gly Ser Ser Tyr
        290 295 300
Pro Ala Asp Val Leu Asn Phe Ala Lys Ile Ile Glu Ser Gly Gln Gly
305 310 315 320
Asn Leu Pro Ala
```

This protein or polypeptide (also referred to herein as “HopPtoP”) has a predicted molecular mass of about 32 kDa and an isoelectric point of about 4.13. Like other hypersensitive response elicitors, the above protein is glycine rich (˜15%), lacks cysteine, is sensitive to proteases, is temperature stable, is secreted via a type III secretion system (where it appears to be targeted to the plant apoplast), and is capable of inducing a hypersensitive response following infiltration onto plant tissues of non-host plants. Like HrpW, HopPtoP has a striking 2-domain structure with the N-terminal portion being “harpin-like” (aa 1-189) and the second portion having homology to lytic transglycosylate (aa 190-324). The harpin domain of HopPtoP possess approximately 53% similarity and 34% identity to the corresponding harpin domain of HrpW from Pseudomonas syringae from DC3000. The lytic transglycosylate domain of HopPtoP shares about 54% similarity to a lytic transglycosylase of Mezorhizobium sp. Based on these similarities, HopPtoP is considered a homolog of HrpW.

The present invention also relates to an isolated DNA molecule having the nucleotide sequence of SEQ ID NO: 3 as follows:

| | | | | | | |
|---|---|---|---|---|---|---|
| atgaatacga | tcaacagaaa | catctacccc | gtctccggga | tttctgcgca | ggatgcccct | 60 |
| gtacaaactg | atcagctcca | gccgcaaggc | cagggcatca | ggccggggca | caatagcaac | 120 |
| ctgatcgact | tcggactgat | acagcaggcc | aatggtccgc | actcatcgct | gaacacatcg | 180 |
| agctccagaa | ttcagccgac | tgacaccagc | acatcctcaa | acaggctggg | gggtaatggc | 240 |
| gatcagttac | tgaacaaact | cgtggaagcg | atccgtaata | tcctcaacaa | cctgctctct | 300 |
| ctgctggaag | gcaatcaaca | ccagggctct | tcgcctgcac | agacccagcg | tgaacagacg | 360 |
| ccgacgtcca | ctcaatcgca | cgcttcgcct | tcctcgtcgt | cttcatcttc | gccgtcgaca | 420 |
| tcctcccagt | cttcaccctc | agtgccttca | acgcctcagg | gcaacgcaga | aaaaccgttt | 480 |
| gtggtgcaga | gcgatcatcc | ggcggaaaaa | ccggtatcgc | tgcagagaac | ctcagagcca | 540 |
| acgtctgtga | cgccgccaca | aacaccaccg | caggctgtcg | agcgaaacag | cattaccccg | 600 |
| gacaaggcac | cggccaaacc | cgaagcggta | aagccggcag | tggtcaacga | cccggtgctg | 660 |
| ccgaaaacct | cgatccctgc | cgccgccaag | cctgacagca | cggtgaccgc | cgcaaaacac | 720 |
| gcgacgcccg | ctgcccgtgg | ccagggcgct | gacatgtccg | gcatgatcgg | ttttgccaag | 780 |
| gaagccaata | ccaccggggg | caacaacggc | gaagtggtca | ccgtgaacac | ggttgccgac | 840 |
| ctcaagaagt | acatggagga | cgacaaagcc | cgcaccgtca | agctgggggC | caacctgtct | 900 |
| gccgacagta | aagtgtcgat | aaatttcggg | gccaacaaaa | ccctgctggg | caccgataaa | 960 |
| ggcaacaccc | tgcacaacat | ctatctggcc | agcggcaaga | ccgccagcaa | cgacattttc | 1020 |
| cagaatctga | acttcaacca | cgacgcccgt | taccgtgaaa | acggcgacat | gcagatgttc | 1080 |
| atcagcagcg | gtcagaaata | ctggatcgac | cacatcaccg | ctaccggaac | caaggatcag | 1140 |
| aaccccaaag | gtctggataa | actgctctac | gtgggcggca | aggcagataa | cgtcagcctg | 1200 |
| accaattcga | aattccagaa | caacgagtat | ggcgtgattc | tcggtcagcc | ggacgactcg | 1260 |
| gcagccgcca | aagccgagta | caagggctac | ccacggatga | caatcgccaa | caacgtgttc | 1320 |
| agcaacctcg | atgtccgcgg | gcccggtctg | tttcgtcagg | gccaatttga | cgtagttaac | 1380 |
| aactcgatcg | acaaattcca | cctcggtttc | actgcgaccg | ggaacgctac | catcctgtcg | 1440 |
| caggccaact | atttcagcaa | cggtgtcgat | gtttccaaca | aggcaagtaa | tagcggcgtg | 1500 |
| ctggatgact | acggcgatgc | gcacttcaaa | gacatcggca | gtaacgtcag | tttcactcag | 1560 |
| aaatcgccgg | ttaccgcctg | gacaccgagc | tacaaccggg | acgtgaaaac | agccgaagca | 1620 |
| gccagagcct | atgacctggc | caatgcgggt | gcacaggtcg | tgaaataa | | 1668 |

This DNA molecule of the present invention encodes a protein or polypeptide having the amino acid sequence of SEQ ID NO: 4 as follows:

```
Met Asn Thr Ile Asn Arg Asn Ile Tyr Pro Val Ser Gly Ile Ser Ala
    1 5 10 25
Gln Asp Ala Pro Val Gln Thr Asp Gln Leu Gln Pro Gln Gly Gln Gly
                          20 25 30
Ile Arg Pro Gly His Asn Ser Asn Leu Ile Asp Phe Gly Leu Ile Gln
                  35 40 45
Gln Ala Asn Gly Pro His Ser Ser Leu Asn Thr Ser Ser Ser Arg Ile
          50 55 60
Gln Pro Thr Asp Thr Ser Thr Ser Ser Asn Arg Leu Gly Gly Asn Gly
  65 70 75 80
Asp Gln Leu Leu Asn Lys Leu Val Glu Ala Ile Arg Asn Ile Leu Asn
                                  85 90 95
Asn Leu Leu Ser Leu Leu Glu Gly Asn Gln His Gln Gly Ser Ser Pro
                        100 105 110
Ala Gln Thr Gln Arg Glu Gln Thr Pro Thr Ser Thr Gln Ser His Ala
                115 120 125
Ser Pro Ser Ser Ser Ser Ser Ser Ser Pro Ser Thr Ser Ser Gln Ser
        130 135 140
Ser Pro Ser Val Pro Ser Thr Pro Gln Gly Asn Ala Glu Lys Pro Phe
145 150 155 160
Val Val Gln Ser Asp His Pro Ala Glu Lys Pro Val Ser Leu Gln Arg
                                165 170 175
Thr Ser Gln Pro Thr Ser Val Thr Pro Pro Gln Thr Pro Pro Gln Ala
                        180 185 190
Val Glu Arg Asn Ser Ile Thr Pro Asp Lys Ala Pro Ala Lys Pro Glu
                195 200 205
Ala Val Lys Pro Ala Val Val Asn Asp Pro Val Leu Pro Lys Thr Ser
        210 215 220
Ile Pro Ala Ala Ala Lys Pro Asp Ser Thr Val Thr Ala Ala Lys His
225 230 235 240
Ala Thr Pro Ala Ala Arg Gly Gln Gly Ala Asp Met Ser Gly Met Ile
                                245 250 255
Gly Phe Ala Lys Glu Ala Asn Thr Thr Gly Gly Asn Asn Gly Glu Val
                        260 265 270
Val Thr Val Asn Thr Val Ala Asp Leu Lys Lys Tyr Met Glu Asp Asp
                275 280 285
Lys Ala Arg Thr Val Lys Leu Gly Ala Asn Leu Ser Ala Asp Ser Lys
        290 295 300
Val Ser Ile Asn Phe Gly Ala Asn Lys Thr Leu Leu Gly Thr Asp Lys
305 310 315 320
Gly Asn Thr Leu His Asn Ile Tyr Leu Ala Ser Gly Lys Thr Ala Ser
                                325 330 335
Asn Asp Ile Phe Gln Asn Leu Asn Phe Asn His Asp Ala Arg Tyr Arg
                        340 345 350
Glu Asn Gly Asp Met Gln Met Phe Ile Ser Ser Gly Gln Lys Tyr Trp
                355 360 365
Ile Asp His Ile Thr Ala Thr Gly Thr Lys Asp Gln Asn Pro Lys Gly
        370 375 380
Leu Asp Lys Leu Leu Tyr Val Gly Gly Lys Ala Asp Asn Val Ser Leu
385 390 395 400
Thr Asn Ser Lys Phe Gln Asn Asn Glu Tyr Gly Val Ile Leu Gly Gln
                                405 410 415
Pro Asp Asp Ser Ala Ala Ala Lys Ala Glu Tyr Lys Gly Tyr Pro Arg
                        420 425 430
Met Thr Ile Ala Asn Asn Val Phe Ser Asn Leu Asp Val Arg Gly Pro
                435 440 445
Gly Leu Phe Arg Gln Gly Gln Phe Asp Val Val Asn Asn Ser Ile Asp
        450 455 460
Lys Phe His Leu Gly Phe Thr Ala Thr Gly Asn Ala Thr Ile Leu Ser
465 470 475 480
Gln Ala Asn Tyr Phe Ser Asn Gly Val Asp Val Ser Asn Lys Ala Ser
                                485 490 495
Asn Ser Gly Val Leu Asp Asp Tyr Gly Asp Ala His Phe Lys Asp Ile
                        500 505 510
Gly Ser Asn Val Ser Phe Thr Gln Lys Ser Pro Val Thr Ala Trp Thr
                515 520 525
Pro Ser Tyr Asn Arg Asp Val Lys Thr Ala Glu Ala Ala Arg Ala Tyr
        530 535 540
Asp Leu Ala Asn Ala Gly Ala Gln Val Val Lys
545 550 555
```

This protein or polypeptide (also referred to herein as “HopPmaH_{Pto}”) has a predicted molecular mass of about 59 kDa and an isoelectric point of about 7.65. Like other hypersensitive response elicitors, the above protein is glycine rich (˜7.5%), lacks cysteine, is sensitive to proteases, is temperature stable, is secreted via a type III secretion system, and is capable of inducing a hypersensitive response following infiltration onto plant tissues of non-host plants. Like HrpW, HopPmaH_{Pto} has a striking 2-domain structure with the N-terminal portion (aa 1-279) being “harpin-like” and the second portion (aa 280-555) having homology to pectin/pectate lyase. The pectate-lyase domain is about 60% similar and about 43% identical to the pectate/pectin lyase from Bacillus subtilis (Accession No. AF027868, which is hereby incorporated by reference in its entirety). Within the lyase domain, HopPmaH_{Pto} is overall about 30% similar and 22% identical to the pectate/pectin lyase from B. subtilis.

Fragments of the above hypersensitive response elicitor polypeptides or proteins are encompassed by the present invention. Suitable fragments can include those portions of the harpin proteins that contain the harpin-like domain.

Suitable fragments can be produced by several means. In the first approach, subclones of the gene encoding the elicitor protein of the present invention are produced by conventional molecular genetic manipulation by subcloning gene fragments. The subclones then are expressed in vitro or in vivo in bacterial cells to yield a smaller protein or peptide that can be tested for elicitor activity according to the procedure described below, e.g., in Wei et al., Science 257:85-86 (1992), which is hereby incorporated by reference in its entirety.

As an alternative approach, fragments of an elicitor protein can be produced by digestion of a full-length elicitor protein with proteolytic enzymes like chymotrypsin or Staphylococcus proteinase A, or trypsin. Different proteolytic enzymes are likely to cleave elicitor proteins at different sites based on the amino acid sequence of the elicitor protein. Some of the fragments that result from proteolysis may be active elicitors of the hypersensitive response.

In another approach, based on knowledge of the primary structure of the protein, fragments of the elicitor protein gene may be synthesized by using the PCR technique together with specific sets of primers chosen to represent particular portions of the protein. These then would be cloned into an appropriate vector for increased expression of a truncated peptide or protein.

Chemical synthesis can also be used to make suitable fragments. Such a synthesis is carried out using known amino acid sequences for the elicitor being produced. Alternatively, subjecting a full length elicitor to high temperatures and pressures will produce fragments. These fragments can then be separated by conventional procedures (e.g., chromatography, SDS-PAGE).

Variants may also (or alternatively) be modified by, for example, the deletion or addition of amino acids that have minimal influence on the properties, secondary structure and hydropathic nature of the polypeptide. For example, a polypeptide may be conjugated to a signal (or leader) sequence at the N-terminal end of the protein which co-translationally or post-translationally directs transfer of the protein. The polypeptide may also be conjugated to a linker or other sequence for ease of synthesis, purification, or identification of the polypeptide.

Also suitable as isolated nucleic acid molecules according to the present invention is a nucleic acid which has a nucleotide sequence that is at least about 55% similar, preferably at least about 65% similar or more preferably at least about 75% similar, to the nucleotide sequence of SEQ ID NO: 1 or SEQ ID NO: 3 by basic BLAST using default parameters analysis. Even more preferred is that such a nucleotide sequence have a nucleic acid identity to the nucleotide sequence of SEQ ID NO: 1 or SEQ ID NO: 3, either by basic BLAST or ClustalW using default parameters analysis, that is at least about 40%, more preferably at least about 50%, and even more preferably at least about 60%. Higher percentages of identity and/or similarity are even more preferred, such as at least about 80% identity and/or at least about 85% similarity.

Also suitable as an isolated nucleic acid according to the present invention is an isolated nucleic acid molecule that hybridizes to the nucleotide sequence of SEQ ID NO: 1 (or its complement) or SEQ ID NO: 3 (or its complement) under suitably stringent hybridization conditions. Exemplary stringent conditions include the use of a hybridization medium or buffer that contains 5×SSC buffer at a temperature of about 42°-65° C., with hybridization being carried out for about 18-20 hours. Stringency can, of course, be increased by lowering the salt concentration or increasing the temperature at which hybridization occurs. Thus, in another embodiment, the hybridization temperature is between about 52°-60° C. In yet another embodiment, the hybridization is between 55°-57° C. Another example of suitable high stringency conditions is when hybridization is carried out at 65° C. for 20 hours in a medium containing 1M NaCl, 50 mM Tris-HCl, pH 7.4, 10 mM EDTA, 0.1% sodium dodecyl sulfate, 0.2% ficoll, 0.2% polyvinylpyrrolidone, 0.2% bovine serum albumin, 50 μm g/ml E. coli DNA. Wash conditions can be selected at varying stringency requirements, as long as the wash conditions are suitable to remove non-specifically bound nucleic acid molecules. Typically, when nucleic acid molecules of longer than about 200 bases are used as probes in hybridization protocols (such as radiolabeled DNA molecules of SEQ ID NO: 1 or SEQ ID NO: 3), the importance of the wash conditions is minimized. See Sambrook et al., Molecular Cloning: A Laboratory Manual, Second Edition, Cold Spring Harbor Press, NY (1989), which is hereby incorporated by reference in its entirety. Thus, a series of increasingly stringent wash conditions can be performed, beginning with lower stringency conditions and continuing until the stringency conditions of the hybridization procedure is achieved. The series of washes can be performed for suitable time periods, typically anywhere from about 15 minutes up to about 2 hours, although shorter or longer washes are certainly effective. However, any DNA molecules hybridizing to the DNA molecule of SEQ ID NO: 1 (or its complement) or SEQ ID NO: 3 (or its complement) under such stringent conditions must not be identical to the nucleic acids encoding the HrpW hypersensitive response elicitor proteins or polypeptides of Erwinia amylovora (see Kim et al., J. Bacteriol. 180(19):5203-5210 (1998), which is hereby incorporated by reference in its entirety) or Pseudomonas syringae pv. tomato (see Charkowski et al., J. Bacteriol. 180:5211-5217 (1998), which is hereby incorporated by reference in its entirety).

The protein or polypeptide of the present invention is preferably produced in purified form (preferably at least about 80%, more preferably 90%, pure) by conventional techniques. Typically, the protein or polypeptide of the present invention is secreted into the growth medium of recombinant host cells. Such secretion can be performed in accordance with the protocol established in PCT Application Publ. No. WO 00/02996 to Bauer et al., which is hereby incorporated by reference in its entirety. Alternatively, the protein or polypeptide of the present invention is produced but not secreted into growth medium. In such cases, to isolate the protein, the host cell (e.g., E. coli) carrying a recombinant plasmid is propagated, lysed by sonication, heat, differential pressure, or chemical treatment, and the homogenate is centrifuged to remove bacterial debris. The supernatant is then subjected to sequential ammonium sulfate precipitation. The fraction containing the polypeptide or protein of the present invention is subjected to gel filtration in an appropriately sized dextran or polyacrylamide column to separate the proteins. If necessary, the protein fraction may be further purified by high performance liquid chromatography (“HPLC”).

The DNA molecule encoding the hypersensitive response elicitor polypeptide or protein can be incorporated in cells using conventional recombinant DNA technology. Generally, this involves inserting the DNA molecule into an expression system to which the DNA molecule is heterologous (i.e., not normally present). The heterologous DNA molecule is inserted into the expression system or vector in proper sense orientation and correct reading frame. The vector contains the necessary elements for the transcription and translation of the inserted protein-coding sequences. As discussed more fully below, the elements for transcription and translation differ depending on the type of system being employed (e.g., eukaryotic vs. prokaryotic, and plant vs. animal).

The present invention also relates to an expression vector containing a DNA molecule encoding a hypersensitive response elicitor protein. The nucleic acid molecule of the present invention may be inserted into any of the many available expression vectors using reagents that are well known in the art. In preparing a DNA vector for expression, the various DNA sequences may normally be inserted or substituted into a bacterial plasmid. Any convenient plasmid may be employed, which will be characterized by having a bacterial replication system, a marker which allows for selection in a bacterium, and generally one or more unique, conveniently located restriction sites. Numerous plasmids, referred to as transformation vectors, are available for plant transformation. The selection of a vector will depend on the preferred transformation technique and target species for transformation.

A variety of vectors are available for stable transformation using Agrobacterium tumefaciens, a soilborne bacterium that causes crown gall. Crown gall is characterized by tumors or galls that develop on the lower stem and main roots of the infected plant. These tumors are due to the transfer and incorporation of part of the bacterium plasmid DNA into the plant chromosomal DNA. This transfer DNA (T-DNA) is expressed along with the normal genes of the plant cell. The plasmid DNA, pTI, or Ti-DNA, for “tumor inducing plasmid,” contains the vir genes necessary for movement of the T-DNA into the plant chromosomal DNA. The T-DNA carries genes that encode proteins involved in the biosynthesis of plant regulatory factors, and bacterial nutrients (opines). The T-DNA is delimited by two 25 bp imperfect direct repeat sequences called the “border sequences.” By removing the oncogene and opine genes, and replacing them with a gene of interest, it is possible to transfer foreign DNA into the plant without the formation of tumors or the multiplication of Agrobacterium tumefaciens. See Fraley et al., Proc. Nat'l Acad. Sci., 80:4803-4807 (1983), which is hereby incorporated by reference in its entirety.

Other suitable vectors for practicing the present invention include, but are not limited to, the following viral vectors such as lambda vector system gt11, gtWES.tB, Charon 4, and plasmid vectors such as pBR322, pBR325, pACYC177, pACYC184, pUC8, pUC9, pUC18, pUC19, pLG339, pR290, pKC37, pKC101, SV 40, pBluescript II SK +/− or KS +/− (see “Stratagene Cloning Systems” Catalog (1993) from LaJolla, Calif., which is hereby incorporated by reference in its entirety), pQE, pIH821, pGEX, pET series (Studier et al., Methods in Enzymology. 185:60-89 (1990) which is hereby incorporated by reference in its entirety), and any derivatives thereof. Any appropriate vectors now known or later described for genetic transformation are suitable for use with the present invention. Recombinant molecules can be introduced into cells via transformation, particularly transduction, conjugation, mobilization, or electroporation. The DNA sequences are cloned into the vector using standard cloning procedures in the art as described by Sambrook et al., Molecular Cloning: A Laboratory Manual, Cold Springs Laboratory, Cold Springs Harbor, N.Y. (1989), which is hereby incorporated by reference in its entirety.

U.S. Pat. No. 4,237,224 to Cohen and Boyer, which is hereby incorporated by reference in its entirety, describes the production of expression systems in the form of recombinant plasmids using restriction enzyme cleavage and ligation with DNA ligase. These recombinant plasmids are then introduced by means of transformation and replicated in unicellular cultures including prokaryotic organisms and eukaryotic cells grown in tissue culture.

Recombinant genes may also be introduced into viruses, such as vaccinia virus. Recombinant viruses can be generated by transfection of plasmids into cells infected with virus.

A variety of host-vector systems may be utilized to express the protein-encoding sequence(s). Primarily, the vector system must be compatible with the host cell used. Host-vector systems include but are not limited to the following: bacteria transformed with bacteriophage DNA, plasmid DNA, or cosmid DNA; microorganisms such as yeast containing yeast vectors; mammalian cell systems infected with virus (e.g., vaccinia virus, adenovirus, etc.); insect cell systems infected with virus (e.g., baculovirus); and plant cells infected by bacteria. The expression elements of these vectors vary in their strength and specificities. Depending upon the host-vector system utilized, any one of a number of suitable transcription and translation elements can be used.

Different genetic signals and processing events control many levels of gene expression (e.g., DNA transcription and messenger RNA (mRNA) translation).

Transcription of DNA is dependent upon the presence of a promoter which is a DNA sequence that directs the binding of RNA polymerase and thereby promotes mRNA synthesis. The DNA sequences of eukaryotic promoters differ from those of prokaryotic promoters. Furthermore, eukaryotic promoters and accompanying genetic signals may not be recognized in or may not function in a prokaryotic system, and, further, prokaryotic promoters are not recognized and do not function in eukaryotic cells.

Similarly, translation of mRNA in prokaryotes depends upon the presence of the proper prokaryotic signals which differ from those of eukaryotes. Efficient translation of mRNA in prokaryotes requires a ribosome binding site called the Shine-Dalgarno (“SD”) sequence on the mRNA. This sequence is a short nucleotide sequence of mRNA that is located before the start codon, usually AUG, which encodes the amino-terminal methionine of the protein. The SD sequences are complementary to the 3′-end of the 16S rRNA (ribosomal RNA) and probably promote binding of mRNA to ribosomes by duplexing with the rRNA to allow correct positioning of the ribosome. For a review on maximizing gene expression, see Roberts and Lauer, Methods in Enzymology, 68:473 (1979), which is hereby incorporated by reference in its entirety.

Promoters vary in their “strength” (i.e., their ability to promote transcription). For the purposes of expressing a cloned gene, it is desirable to use strong promoters in order to obtain a high level of transcription and, hence, expression of the gene. Depending upon the host cell system utilized, any one of a number of suitable promoters may be used. For instance, when cloning in E. coli, its bacteriophages, or plasmids, promoters such as the T7 phage promoter, lac promoter, trp promoter, recA promoter, ribosomal RNA promoter, the P_{R} and P_{L} promoters of coliphage lambda and others, including but not limited to lacUV5, ompF, bla, lpp, and the like, may be used to direct high levels of transcription of adjacent DNA segments. Additionally, a hybrid trp-lacUV5 (tac) promoter or other E. coli promoters produced by recombinant DNA or other synthetic DNA techniques may be used to provide for transcription of the inserted gene.

Bacterial host cell strains and expression vectors may be chosen which inhibit the action of the promoter unless specifically induced. In certain operations, the addition of specific inducers is necessary for efficient transcription of the inserted DNA. For example, the lac operon is induced by the addition of lactose or IPTG (isopropylthio-beta-D-galactoside). A variety of other operons, such as trp, pro, etc., are under different controls.

Specific initiation signals are also required for efficient gene transcription and translation in prokaryotic cells. These transcription and translation initiation signals may vary in “strength” as measured by the quantity of gene specific messenger RNA and protein synthesized, respectively. The DNA expression vector, which contains a promoter, may also contain any combination of various “strong” transcription and/or translation initiation signals. For instance, efficient translation in E. coli requires an SD sequence about 7-9 bases 5′ to the initiation codon (“ATG”) to provide a ribosome binding site. Thus, any SD-ATG combination that can be utilized by host cell ribosomes may be employed. Such combinations include but are not limited to the SD-ATG combination from the cro gene or the Ngene of coliphage lambda, or from the E. coli tryptophan E, D, C, B or A genes. Additionally, any SD-ATG combination produced by recombinant DNA or other techniques involving incorporation of synthetic nucleotides may be used.

In one aspect of the present invention, the nucleic acid molecule of the present invention is incorporated into an appropriate vector in the sense direction, such that the open reading frame is properly oriented for the expression of the encoded protein under control of a promoter of choice. This involves the inclusion of the appropriate regulatory elements into the DNA-vector construct. These include non-translated regions of the vector, useful promoters, and 5′ and 3′ untranslated regions which interact with host cellular proteins to carry out transcription and translation. Such elements may vary in their strength and specificity. Depending on the vector system and host utilized, any number of suitable transcription and translation elements, including constitutive and inducible promoters, may be used.

A constitutive promoter is a promoter that directs expression of a gene throughout the development and life of an organism. Examples of some constitutive promoters that are widely used for inducing expression of transgenes include, without limitation, the nopoline synthase (NOS) gene promoter from Agrobacterium tumefaciens (U.S. Pat. No. 5,034,322 to Rogers et al., which is hereby incorporated by reference in its entirety), the cauliflower mosaic virus (CaMv) 35S and 19S promoters (U.S. Pat. No. 5,352,605 to Fraley et al., which is hereby incorporated by reference in its entirety), those derived from any of the several actin genes, which are known to be expressed in most cells types (U.S. Pat. No. 6,002,068 to Privalle et al., which is hereby incorporated by reference in its entirety), the ubiquitin promoter, which is a gene product known to accumulate in many cell types, the enhanced 35S promoter described in U.S. Pat. No. 5,106,739 to Comai et al. (which is hereby incorporated by reference in its entirety), the dual 35S promoter, the FMV promoter from figwort mosaic virus that is described in U.S. Pat. No. 5,378,619 to Rogers et al. (which is hereby incorporated by reference in its entirety), the RIT-DNA promoter described in U.S. Pat. No. 5,466,792 to Slightom et al. (which is hereby incorporated by reference in its entirety), the octopine T-DNA promoter described in U.S. Pat. No. 5,428,147 to Barker et al. (which is hereby incorporated by reference in its entirety), the alcohol dehydrogenase 1 promoter (Callis et al., Genes Dev., 1(10):1183-1200 (1987), which is hereby incorporated by reference in its entirety), the patatin promoter B33 (Rocha-Sosa et al., EMBO J., 8:23-29 (1989), which is hereby incorporated by reference in its entirety), the E8 promoter (Deikman et al., EMBO J., 7(11):3315-3320 (1988), which is hereby incorporated by reference in its entirety), the beta-conglycin promoter (Tierney et al., Planta, 172:356-363 (1987), which is hereby incorporated by reference in its entirety), the acid chitinase promoter (Samac et al., Plant Physiol., 93:907-914 (1990), which is hereby incorporated by reference in its entirety), the Arabidopsis histone H4 promoter described in U.S. Pat. No. 5,491,288 to Chaubet et al. (which is hereby incorporated by reference in its entirety), or the recombinant promoter for expression of genes in monocots described in U.S. Pat. No. 5,290,924 to Last et al. (which is hereby incorporated by reference in its entirety).

An inducible promoter is a promoter that is capable of directly or indirectly activating transcription of one or more DNA sequences or genes in response to an inducer. In the absence of an inducer, the DNA sequences or genes will not be transcribed. The inducer can be a chemical agent, such as a metabolite, growth regulator, herbicide or phenolic compound, or a physiological stress directly imposed upon the plant such as cold, heat, salt, toxins, or through the action of a pathogen or disease agent such as a virus or fungus. A plant cell containing an inducible promoter may be exposed to an inducer by externally applying the inducer to the cell or plant such as by spraying, watering, heating, or by exposure to the operative pathogen. In addition, inducible promoters include promoters that function in a tissue specific manner to regulate the gene of interest within selected tissues of the plant Examples of such tissue specific promoters include seed, flower, or root specific promoters as are well known in the field (U.S. Pat. No. 5,750,385 to Shewmaker et al., which is hereby incorporated by reference in its entirety).

In one aspect of the present invention, the inducible promoter is a pathogen-inducible promoter. Such promoters include those from pathogenesis-related proteins (e.g., PR proteins, SAR proteins, beta-1,3-glucanase, chitinase), which are induced following infection by a pathogen (see, e.g., Redolfi et al., Neth. J. Plant Pathol. 89:245-254 (1983); Uknes et al., Plant Cell 4:645-656 (1992); and Van Loon, Plant Mol. Virol. 4:111-116 (1985), which are hereby incorporated by reference in their entirety). Another aspect of the present invention involves promoters that are expressed locally at or near the site of pathogen infection (see, e.g., Marineau et al., Plant Mol. Biol. 9:335-342 (1987); Matton et al., Molecular Plant-Microbe Interactions 2:325-331 (1989); Somsisch et al., Proc. Natl. Acad. Sci. U.S.A. 83:2427-2430 (1986); Somsisch et al., Molecular and General Genetics 2:93-98 (1988); and Yang, Proc. Natl. Acad. Sci. U.S.A. 93:14972-14977 (1996), which are hereby incorporated by reference in their entirety). See also Chen et al., Plant J. 10:955-966 (1996); Zhang and Sing, Proc. Natl. Acad. Sci. U.S.A. 91:2507-2511 (1994); Warner et al., Plant J. 3:191-201 (1993); Siebertz et al., Plant Cell 1:961-968 (1989), which are hereby incorporated by reference in their entirety).

Additionally, as pathogens find entry into plants through wounds or insect damage, a wound inducible promoter may be used in the construct of the invention. Such wound inducible promoters include potato proteinase inhibitor (pin II) gene (Ryan, Ann. Rev. Phytopath. 28:425-449 (1990); Duan et al., Nature Biotechnology 14:494-498 (1996), which are hereby incorporated by reference in its entirety); wun1 and wun2, U.S. Pat. No. 5,428,148 to Reddy et al. (which is hereby incorporated by reference in its entirety); win1 and win2 (Stanford et al., Mol. Gen. Genet. 215:200-208 (1989), which is hereby incorporated by reference in its entirety); systemin (McGurl et al., Science 255:1570-1573 (1992), which is hereby incorporated by reference in its entirety); WIP1 (Rohrmeier et al., Plant Mol Biol 22:783-792 (1993); Eckelkamp et al., FEBS Letters 323:73-76 (1993), which are hereby incorporated by reference in their entirety); and MPI gene (Cordero et al., Plant Journal 6(2): 141-150 (1994), which is hereby incorporated by reference in its entirety).

The DNA construct of the present invention also includes an operable 3′ regulatory region, selected from among those which are capable of providing correct transcription termination and polyadenylation of mRNA for expression in the host cell of choice, operably linked to a DNA molecule which encodes for a protein of choice. A number of 3′ regulatory regions are known to be operable in plants. Exemplary 3′ regulatory regions include, without limitation, the nopaline synthase 3′ regulatory region (Fraley et al., Proc. Nat'l Acad. Sci. USA 80:4803-4807 (1983), which is hereby incorporated by reference in its entirety) and the cauliflower mosaic virus 3′ regulatory region (Odell et al., Nature 313(6005):810-812 (1985), which is hereby incorporated by reference in its entirety). Virtually any 3′ regulatory region known to be operable in plants would suffice for proper expression of the coding sequence of the DNA construct of the present invention.

The vector of choice, promoter, and an appropriate 3′ regulatory region can be ligated together to produce the DNA construct of the present invention using well known molecular cloning techniques as described in Sambrook et al., Molecular Cloning: A Laboratory Manual, Second Edition, Cold Spring Harbor Press, NY (1989), and Ausubel et al., Current Protocols in Molecular Biology, John Wiley & Sons, New York, N.Y. (1989), which are hereby incorporated by reference in their entirety.

Once the isolated DNA molecule encoding the hypersensitive response elicitor polypeptide or protein has been cloned into an expression system, it is ready to be incorporated into a host cell. Such incorporation can be carried out by the various forms of transformation noted above, depending upon the vector/host cell system. Suitable host cells include, but are not limited to, bacteria, virus, yeast, mammalian cells, insect, plant, and the like.

The present invention further relates to methods of imparting disease resistance, stress resistance, and enhanced growth to plants or plants grown from treated seeds, controlling insects on plants or plants grown from treated plant seeds, imparting post-harvest disease or desiccation resistance in fruits or vegetables, imparting enhanced longevity of fruit or vegetable ripeness, imparting desiccation resistance to cuttings of ornamental plants, and/or promoting early flowering of ornamental plants. These methods involve applying a hypersensitive response elicitor polypeptide or protein in a non-infectious form to all or part of a plant, plant seed, cutting, and/or fruit or vegetable under conditions where the polypeptide or protein contacts all or part of the cells of the plant, plant seed, cutting, and/or fruit or vegetable. Alternatively, the hypersensitive response elicitor protein or polypeptide can be applied to plants such that seeds recovered from such plants themselves are able to impart disease resistance, stress resistance, and enhanced growth to plants grown from those seeds, to control insects on plants grown from those seeds, to impart post-harvest disease or desiccation resistance in fruits or vegetables harvested from plants grown from those seeds, to impart enhanced longevity of fruit or vegetable ripeness for fruits or vegetables harvested from plants grown from those seeds, to impart desiccation resistance to cuttings of ornamental plants grown from those seeds, and/or promote early flowering of ornamental plants grown from those seeds.

Application of the hypersensitive response elicitor polypeptide or protein in non-infectious form can be carried out in a number of ways including, without limitation: 1) application of an isolated elicitor polypeptide or protein; 2) application of bacteria which do not cause disease and are transformed with genes encoding a hypersensitive response elicitor polypeptide or protein; and 3) application of bacteria which cause disease in some plant species (but not in those to which they are applied) and naturally contain a gene encoding the hypersensitive response elicitor polypeptide or protein.

In one embodiment of the present invention, the hypersensitive response elicitor polypeptide or protein of the present invention can be isolated directly from Pseudomonas syringae pv. tomato as described in the Examples infra. Preferably, however, the isolated hypersensitive response elicitor polypeptide or protein of the present invention is produced recombinantly and purified as described supra.

In other embodiments of the present invention, the hypersensitive response elicitor polypeptide or protein of the present invention can be applied to plants or plant seeds by applying bacteria containing genes encoding the hypersensitive response elicitor polypeptide or protein. Such bacteria must be capable of secreting or exporting the polypeptide or protein so that the elicitor can contact plant or plant seed cells. In these embodiments, the hypersensitive response elicitor polypeptide or protein is produced by the bacteria in planta or on seeds or just prior to introduction of the bacteria to the plants or plant seeds.

In one embodiment of the bacterial application mode of the present invention, the bacteria do not cause the disease and have been transformed (e.g., recombinantly) with genes encoding a hypersensitive response elicitor polypeptide or protein. For example, E. coli, which does not elicit a hypersensitive response in plants, can be transformed with genes encoding a hypersensitive response elicitor polypeptide or protein and optionally type III secretion systems (U.S. patent application Ser. No. 09/350,852 to Bauer et al., filed Jul. 9, 1999, which is hereby incorporated by reference in its entirety), and then applied to plants. Bacterial species other than E. coli can also be used in this embodiment of the present invention.

In another embodiment of the bacterial application mode of the present invention, the bacteria do cause disease and naturally contain a gene encoding a hypersensitive response elicitor polypeptide or protein. Examples of such bacteria are noted above. However, in this embodiment, these bacteria are applied to plants or their seeds which are not susceptible to the disease carried by the bacteria For example, Pseudomonas syringae pv. tomato causes disease in tomato but not in beans. However, such bacteria will elicit a hypersensitive response in beans. Accordingly, in accordance with this embodiment of the present invention, Pseudomonas syringae pv. tomato can be applied to bean plants or seeds to impart disease resistance to plants, enhance plant growth, and control insects on plants or plants grown from the plant seeds, impart stress resistance to plants, and/or impart post-harvest disease or desiccation resistance in fruits or vegetables, without causing disease in that species.

The method of the present invention can be utilized to treat a wide variety of plants, their seeds, their cuttings, and/or their harvested fruit or vegetables to impart disease resistance, stress resistance, and enhanced growth to plants or plants grown from treated seeds, to control insects on plants or plants grown from treated plant seeds, to impart post-harvest disease or desiccation resistance in fruits or vegetables, to impart enhanced longevity of fruit or vegetable ripeness, to impart desiccation resistance to cuttings of ornamental plants, and/or promote early flowering of ornamental plants.

Suitable plants include dicots and monocots. More particularly, useful crop plants can include, without limitation: alfalfa, rice, wheat, barley, rye, cotton, sunflower, peanut, corn, potato, sweet potato, bean, pea, chicory, lettuce, endive, cabbage, brussel sprouts, beet, parsnip, turnip, cauliflower, broccoli, radish, spinach, onion, garlic, eggplant, pepper, celery, carrot, squash, pumpkin, zucchini, cucumber, apple, pear, melon, citrus, strawberry, grape, raspberry, pineapple, soybean, tobacco, tomato, sorghum, and sugarcane. As used herein, ornamental plants are those plants that are not crop plants. Examples of suitable ornamental plants include, without limitation: Arabidopsis thaliana, Saintpaulia, petunia, pelargonium, poinsettia, chrysanthemum, carnation, rose, tulip, and zinnia.

With regard to the use of the hypersensitive response elicitor protein or polypeptide of the present invention in imparting disease resistance, absolute immunity against infection may not be conferred, but the severity of the disease is reduced and symptom development is delayed. Lesion number, lesion size, and extent of sporulation of fungal pathogens are all decreased. This method of imparting disease resistance has the potential for treating previously untreatable diseases, treating diseases systemically which might not be treated separately due to cost, and avoiding the use of infectious agents or environmentally harmful materials.

The method of imparting pathogen resistance to plants in accordance with the present invention is useful in imparting resistance to a wide variety of pathogens including viruses, bacteria, and fungi (see WO 96/39802 to Wei et al. and WO 98/24297 to Qiu et al., which are hereby incorporated by reference in their entirety). Resistance, inter alia, to the following viruses can be achieved by the method of the present invention: Tobacco mosaic virus and Tomato mosaic virus. Resistance, inter alia, to the following bacteria can also be imparted to plants in accordance with present invention: Pseudomonas solancearum, Pseudomonas syringae pv. tabaci, and Xanthamonas campestris pv. pelargonii. Plants can be made resistant, inter alia, to the following fungi by use of the method of the present invention: Fusarium oxysporum and Phytophthora infestans.

With regard to the use of the hypersensitive response elicitor protein or polypeptide of the present invention to enhance plant growth, various forms of plant growth enhancement or promotion can be achieved (WO 98/32844 to Qiu et al., which is hereby incorporated by reference in its entirety). This can occur as early as when plant growth begins from seeds or later in the life of a plant. For example, plant growth according to the present invention encompasses greater yield, increased quantity of seeds produced, increased percentage of seeds germinated, increased plant size, greater biomass, more and bigger fruit, earlier fruit coloration, and earlier fruit and plant maturation. As a result, the present invention provides significant economic benefit to growers. For example, early germination and early maturation permit crops to be grown in areas where short growing seasons would otherwise preclude their growth in that locale. Increased percentage of seed germination results in improved crop stands and more efficient seed use. Greater yield, increased size, and enhanced biomass production allow greater revenue generation from a given plot of land.

Another aspect of the present invention is directed to effecting any form of insect control for plants (WO 98/37752 to Zitter et al., which is hereby incorporated by reference in its entirety). For example, insect control according to the present invention encompasses preventing insects from contacting plants to which the hypersensitive response elicitor has been applied, preventing direct insect damage to plants by feeding injury, causing insects to depart from such plants, killing insects proximate to such plants, interfering with insect larval feeding on such plants, preventing insects from colonizing host plants, preventing colonizing insects from releasing phytotoxins, etc. The present invention also prevents subsequent disease damage to plants resulting from insect infection.

The present invention is effective against a wide variety of insects. European corn borer is a major pest of corn (dent and sweet corn) but also feeds on over 200 plant species including green, wax, and lima beans and edible soybeans, peppers, potato, and tomato plus many weed species. Additional insect larval feeding pests which damage a wide variety of vegetable crops include the following: beet armyworm, cabbage looper, corn ear worm, fall armyworm, diamondback moth, cabbage root maggot, onion maggot, seed corn maggot, pickleworm (melonworm), pepper maggot, and tomato pinworm. Collectively, this group of insect pests represents the most economically important group of pests for vegetable production worldwide.

The present invention relates to the use of the hypersensitive response elicitors of the present invention to impart stress resistance to plants (WO 00/28055 to Wei et al., which is hereby incorporated by reference in its entirety). Resistance can be afforded to a variety of stresses, such as any environmental factor having an adverse effect on plant physiology and development. Examples of such environmental stress include without limitation: climate-related stress (e.g., drought, water, frost, cold temperature, high temperature, excessive light, and insufficient light), air pollution stress (e.g., carbon dioxide, carbon monoxide, sulfur dioxide, NO_{X}, hydrocarbons, ozone, ultraviolet radiation, acidic rain), chemical (e.g., insecticides, fungicides, herbicides, heavy metals), and nutritional stress (e.g., fertilizer, micronutrients, macronutrients).

The present invention is also effective in imparting post-harvest disease or desiccation resistance in fruits or vegetables (WO 01/80639 to Wei et al., which is hereby incorporated by reference in its entirety), as well as imparting desiccation resistance to cuttings of ornamental plants (WO 02/37960 to Wei et al., which is hereby incorporated by reference in its entirety). The present invention also relates to ornamental cuttings that have themselves been treated with a hypersensitive response elicitor protein or polypeptide of the present invention following removal from an ornamental plant, as well as cuttings removed from ornamental plants that have been treated therewith. The present invention also includes fruit or vegetable products that have been treated with a hypersensitive response elicitor protein or polypeptide of the present invention or removed from plants that have been treated therewith.

In addition to imparting post-harvest disease or desiccation resistance, the longevity of fruit or vegetable ripeness can be enhanced (WO 01/80639 to Wei et al., which is hereby incorporated by reference in its entirety). Enhanced ripeness longevity will afford a longer shelf-life to produce and thereby promote less consumer waste.

The method of the present invention involving application of the hypersensitive response elicitor polypeptide or protein can be carried out through a variety of procedures when all or part of the plant is treated, including leaves, stems, roots, propagules (e.g., cuttings), etc. This may (but need not) involve infiltration of the hypersensitive response elicitor polypeptide or protein into the plant. Suitable application methods include high or low pressure spraying, injection, and leaf abrasion proximate to when elicitor application takes place. Seed treatments can also be employed, as described in U.S. Pat. No. 6,235,974 to Qiu et al., which is hereby incorporated by reference in its entirety. When treating plant seeds, in accordance with the application embodiment of the present invention, the hypersensitive response elicitor protein or polypeptide can be applied by low or high pressure spraying, coating, immersion, or injection (U.S. Pat. No. 6,235,974 to Qiu et al., which is hereby incorporated by reference in its entirety). Other suitable application procedures can be envisioned by those skilled in the art provided they are able to effect contact of the hypersensitive response elicitor polypeptide or protein with cells of the plant or plant seed. Once treated with the hypersensitive response elicitor of the present invention, the seeds can be planted in natural or artificial soil and cultivated using conventional procedures to produce plants. After plants have been propagated from seeds treated in accordance with the present invention, the plants may be treated with one or more applications of the hypersensitive response elicitor protein or polypeptide to impart disease resistance, stress resistance, and enhanced growth to plants or plants grown from treated seeds, to control insects on plants or plants grown from treated plant seeds, to impart post-harvest disease or desiccation resistance in fruits or vegetables, to impart enhanced longevity of fruit or vegetable ripeness, to impart desiccation resistance to cuttings of ornamental plants, and/or promote early flowering of ornamental plants.

The hypersensitive response elicitor polypeptide or protein can be applied to plants or plant seeds in accordance with the present invention alone or in a mixture with other materials. Alternatively, the hypersensitive response elicitor polypeptide or protein can be applied separately to plants with other materials being applied at different times.

A composition suitable for treating plants or plant seeds in accordance with the application embodiment of the present invention contains a hypersensitive response elicitor polypeptide or protein in a carrier. Suitable carriers include water, aqueous solutions, slurries, or dry powders. By way of example, one such composition of harpin_{Ea} (3 wt %) is commercially available from Eden Bioscience Corp. under the tradename Messenger®. It is expected that compositions of the proteins of the present invention can be prepared in a manner similar to that which is used for Messenger®.

Although not required, the composition of the present invention may contain additional additives including fertilizer, insecticide, fungicide, nematacide, and mixtures thereof. Suitable fertilizers include (NH₄)₂NO₃. An example of a suitable insecticide is Malathion. Useful fungicides include Captan.

Other suitable additives include buffering agents, wetting agents, coating agents, and abrading agents. These materials can be used to facilitate the process of the present invention. In addition, the hypersensitive response elicitor polypeptide or protein can be applied to plant seeds with other conventional seed formulation and treatment materials, including clays and polysaccharides.

As an alternative to applying a hypersensitive response elicitor polypeptide or protein to plants or plant seeds in order to impart disease resistance, stress resistance, and enhanced growth to plants or plants grown from treated seeds, to control insects on plants or plants grown from treated plant seeds, to impart post-harvest disease or desiccation resistance in fruits or vegetables, to impart enhanced longevity of fruit or vegetable ripeness, to impart desiccation resistance to cuttings of ornamental plants, and/or promote early flowering of ornamental plants, transgenic plants or plant seeds can be utilized (WO 01/95724 to Wei et al., which is hereby incorporated by reference in its entirety). Using transgenic plants involves providing a transgenic plant transformed with a DNA molecule encoding a hypersensitive response elicitor polypeptide or protein of the present invention and growing the plant under conditions effective to permit that DNA molecule to impart disease resistance, stress resistance, and enhanced growth to the transgenic plants, to control insects on the transgenic plants, to impart post-harvest disease or desiccation resistance in fruits or vegetables removed from the transgenic plants, to impart enhanced longevity of fruit or vegetable ripeness, to impart desiccation resistance to cuttings of transgenic ornamental plants, and/or promote early flowering of transgenic ornamental plants. Alternatively, a transgenic plant seed transformed with a DNA molecule encoding a hypersensitive response elicitor polypeptide or protein of the present invention can be provided and planted in soil. A plant is then propagated from the planted seed under conditions effective to permit that DNA molecule to impart disease resistance, stress resistance, and enhanced growth to plants grown from the transgenic seeds, to control insects on plants grown from the transgenic plant seeds, to impart post-harvest disease or desiccation resistance in fruits or vegetables removed from the transgenic plants, to impart enhanced longevity of fruit or vegetable ripeness, to impart desiccation resistance to cuttings of transgenic ornamental plants, and/or promote early flowering of transgenic ornamental plants.

In the alternative embodiment of the present invention involving the use of transgenic plants and transgenic seeds, a hypersensitive response elicitor polypeptide or protein need not but may be applied topically to the transgenic plants or transgenic plant seeds.

The vector described above can be microinjected directly into plant cells by use of micropipettes to transfer mechanically the recombinant DNA. Crossway, Mol. Gen. Genetics, 202:179-85 (1985), which is hereby incorporated by reference in its entirety. The genetic material may also be transferred into the plant cell using polyethylene glycol. Krens et al., Nature, 296:72-74 (1982), which is hereby incorporated by reference in its entirety.

Another approach to transforming plant cells with a gene which imparts resistance to pathogens is particle bombardment (also known as biolistic transformation) of the host cell. This can be accomplished in one of several ways. The first involves propelling inert or biologically active particles at cells. This technique is disclosed in U.S. Pat. Nos. 4,945,050, 5,036,006, and 5,100,792, all to Sanford et al., which are hereby incorporated by reference in their entirety. Generally, this procedure involves propelling inert or biologically active particles at the cells under conditions effective to penetrate the outer surface of the cell and to be incorporated within the interior thereof. When inert particles are utilized, the vector can be introduced into the cell by coating the particles with the vector containing the heterologous DNA. Alternatively, the target cell can be surrounded by the vector so that the vector is carried into the cell by the wake of the particle. Biologically active particles (e.g., dried bacterial cells containing the vector and heterologous DNA) can also be propelled into plant cells.

Yet another method of introduction is fusion of protoplasts with other entities, either minicells, cells, lysosomes or other fusible lipid-surfaced bodies. Fraley et al., Proc. Natl. Acad. Sci. USA, 79:1859-63 (1982), which is hereby incorporated by reference in its entirety.

The DNA molecule may also be introduced into the plant cells by electroporation. Fromm et al., Proc. Natl. Acad. Sci. USA, 82:5824 (1985), which is hereby incorporated by reference in its entirety. In this technique, plant protoplasts are electroporated in the presence of plasmids containing the expression cassette. Electrical impulses of high field strength reversibly permeabilize biomembranes allowing the introduction of the plasmids. Electroporated plant protoplasts reform the cell wall, divide, and regenerate.

Another method of introducing the DNA molecule into plant cells is to infect a plant cell with Agrobacterium tumefaciens or A. rhizogenes previously transformed with the gene. Under appropriate conditions known in the art, the transformed plant cells are grown to form shoots or roots, and develop further into plants. Generally, this procedure involves inoculating the plant tissue with a suspension of bacteria and incubating the tissue for 48 to 72 hours on regeneration medium without antibiotics at 25-28° C.

Agrobacterium is a representative genus of the gram-negative family Rhizobiaceae. Its species are responsible for crown gall (A. tumefaciens) and hairy root disease (A. rhizogenes). The plant cells in crown gall tumors and hairy roots are induced to produce amino acid derivatives known as opines, which are catabolized only by the bacteria. The bacterial genes responsible for expression of opines are a convenient source of control elements for chimeric expression cassettes. In addition, assaying for the presence of opines can be used to identify transformed tissue.

Heterologous genetic sequences can be introduced into appropriate plant cells, by means of the Ti plasmid of A. tumefaciens or the R1 plasmid of A. rhizogenes. The Ti or Ri plasmid is transmitted to plant cells on infection by Agrobacterium and is stably integrated into the plant genome (Schell, Science, 237:1176-83 (1987), which is hereby incorporated by reference in its entirety).

After transformation, the transformed plant cells can be selected (using appropriate selection media to identify transformants) and then regenerated. Plant regeneration from cultured protoplasts is described in Evans et al., Handbook of Plant Cell Cultures, Vol. 1: MacMillan Publishing Co., New York (1983); and Vasil I. R. (ed.), Cell Culture and Somatic Cell Genetics of Plants, Acad. Press, Orlando, Vol. I (1984) and Vol. III (1986), which are hereby incorporated by reference in their entirety. It is known that practically all plants can be regenerated from cultured cells or tissues.

Means for regeneration vary from species to species of plants, but generally a suspension of transformed protoplasts or a petri plate containing transformed explants is first provided. Callus tissue is formed and shoots may be induced from callus and subsequently rooted. Alternatively, embryo formation can be induced in the callus tissue. These embryos germinate as natural embryos to form plants. The culture media will generally contain various amino acids and hormones, such as auxin and cytokinins. It is also advantageous to add glutamic acid and proline to the medium, especially for such species as corn and alfalfa Efficient regeneration will depend on the medium, on the genotype, and on the history of the culture. If these three variables are controlled, then regeneration is usually reproducible and repeatable.

After the expression cassette is stably incorporated in transgenic plants, it can be transferred to other plants by sexual crossing. Any of a number of standard breeding techniques can be used, depending upon the species to be crossed.

Once transgenic plants of this type are produced, the plants themselves can be cultivated in accordance with conventional procedures. Transgenic seeds can, of course, be recovered from the transgenic plants. These seeds can then be planted in the soil and cultivated using conventional procedures to produce transgenic plants.

When transgenic plants and plant seeds are used in accordance with the present invention, they additionally can be treated with the same materials as are used topically to treat the plants and seeds. These other materials, including hypersensitive response elicitors, can be applied to the transgenic plants and plant seeds by the above-noted procedures, including high or low pressure spraying, injection, coating, and immersion. Similarly, after plants have been propagated from the transgenic plant seeds, the plants may be treated with one or more applications of the hypersensitive response elicitor to impart disease resistance, stress resistance, and enhanced growth to plants, to control insects on plants, to impart post-harvest disease or desiccation resistance in fruits or vegetables, to impart enhanced longevity of fruit or vegetable ripeness, to impart desiccation resistance to cuttings of ornamental plants, and/or promote early flowering of ornamental plants.

Such plants may also be treated with conventional plant treatment agents (e.g., insecticides, fertilizers, etc.). The present invention also relates to fruit or vegetables removed from transgenic plants of the present invention as well as cuttings removed from transgenic ornamental plants of the present invention.

In the embodiment of the present invention where transgenic plants or plant seeds are utilized, it should be appreciated that the transgenic plants or plant seeds can include one or more transgenes other than the transgene encoding the hypersensitive response elicitor protein or polypeptide of the present invention. In particular, the use of hypersensitive response elicitors for the purpose of maximizing the benefit of a transgenic trait or overcoming a concomitant yield penalty is disclosed in U.S. patent application Ser. No. 09/880,371 to Wei et al., filed Jun. 13, 2001 (now published), which is hereby incorporated by reference in its entirety.

### EXAMPLES

Each of the Examples set forth below is intended to illustrate the nature of the present invention but is by no means intended to limit its scope.

### Example 1

### Isolation of HopPtoP and HopPmaH_{Pto} DNA Molecules and Preparation of Expression Vectors

hopPtoP was identified by using a reporter transposon to identify genes in the P. s. tomato DC3000 genome that were induced in a HrpL-dependent manner (Fouts et al., Proc. Natl. Acad. Sci. USA 99(4):2275-2280 (2002) and supplemental materials available online, which are hereby incorporated by reference in their entirety). hopPmaH_{Pto} was identified by the bioinformatic approach of scanning the DC3000 genome with a Hidden Markov Model for Hrp promoter sequences (Fouts et al., Proc. Natl. Acad. Sci. USA 99(4):2275-2280 (2002) and supplemental materials available online, which are hereby incorporated by reference in their entirety). The HrpL-dependent expression of hopPmaH_{Pto} was then confirmed by microarray analysis.

The following primers were used to amplify hopPtoP and hopPmaH_{Pto} from P. syringae pv. tomato DC3000 genomic DNA using standard PCR protocols:

hopPtoP Forward Primer

| | | |
|---|---|---|
| caccatgacc atgggtgttt cac | (SEQ ID NO:5) | 23 |

hopPtoP Reverse Primer

| | | |
|---|---|---|
| agcgggtaaa ttgccctgc | (SEQ ID NO:6) | 19 |

hopPmaH_{Pto} Forward Primer

| | | |
|---|---|---|
| caccatgaat acgatcaac | (SEQ ID NO:7) | 19 |

hopPmaH_{Pto} Reverse Primer

| | | |
|---|---|---|
| tttcacgacc tgtgc | (SEQ ID NO:8) | 15 |

Blunt end PCR fragments were generated for use with Invitrogen™ Gateway™ technology in accordance with the manufacturer's instructions. PCR products were cloned into pENTR/SD/D-TOPO® vector using Gateway™ technology. The Gateway™ technology offers a universal cloning process based on the site-specific recombination properties of bacteriophage lambda, and provides a rapid and highly efficient way to move DNA sequences into multiple vector systems for functional analysis and protein expression.

Briefly, the attachment sites for the vector can recombine with each other. Thus, once an entry clone is created with the pENTR cloning kit, the fragment/gene of interest can easily be cloned into numerous other vectors that contain complementary attachment sites with which the pENTR attachment sites can recombine.
pENTR/SD/D-TOPO®:: hopPtoP and pENTR/SD/D-TOPO®:: hopPmaH_{Pto} were used to clone into pET-DEST42, a ‘Gateway-ized’ vector from Invitrogen™. This placed 6×-histidine and V5 epitope tags to the C-terminal ends of hopPtoP and hopPmaH_{Pto}. The resulting plasmids were designated pCPP5098 (hopPtoP-6×His-V5) and pCPP5099 (hopPmaH_{Pto}-6×His-V5). These plasmids were transformed into E. coli BL21 DE3 from Novagen. QIAGEN protein purification protocols were used to optimize purification under native conditions. For HopPtoP, conditions described in Alfano et al. (Molecular Microbiology 19:715-728 (1996), which is hereby incorporated by reference in its entirety) were used for purification and expression. The conditions described in Alfano et al., Molecular Microbiology 19:715-728 (1996), which is hereby incorporated by reference in its entirety, can also be used for purification and expression of HopPmaH_{Pto}. In addition, standard conditions well known in the art for growing E. coli (Sambrook et al., Molecular Cloning: A Laboratory Manual, Second Edition, Cold Spring Harbor Press, NY (1989), which is hereby incorporated by reference in its entirety), as well as for purifying His-tagged proteins (Hainfeld et al., Microsc. Microanal. 8(Supp. 2):832CD (2002); Hochuli et al., J. Chromatogr. 411:177-184 (1987); Hainfeld et al., J. Struct. Biol. 127:185-198 (1999); Buchel et al., J. Mol. Biol. 312:371-379 (2001); Hata et al., J. Virol. Methods 84(2):117-126 (2000); Blanc et al., J. Virol. Methods 77(1):11-15 (1999); and Schmidbauer et al., Biochemica 3:22-24 (1997), which are hereby incorporated by reference in their entirety), can be used to purify and express HopPtoP and HopPmaH_{Pto}.

### Example 2

### Topical Application of the HopPtoP and HopPmaH_{Pto} to Plants and HR Activity Thereof

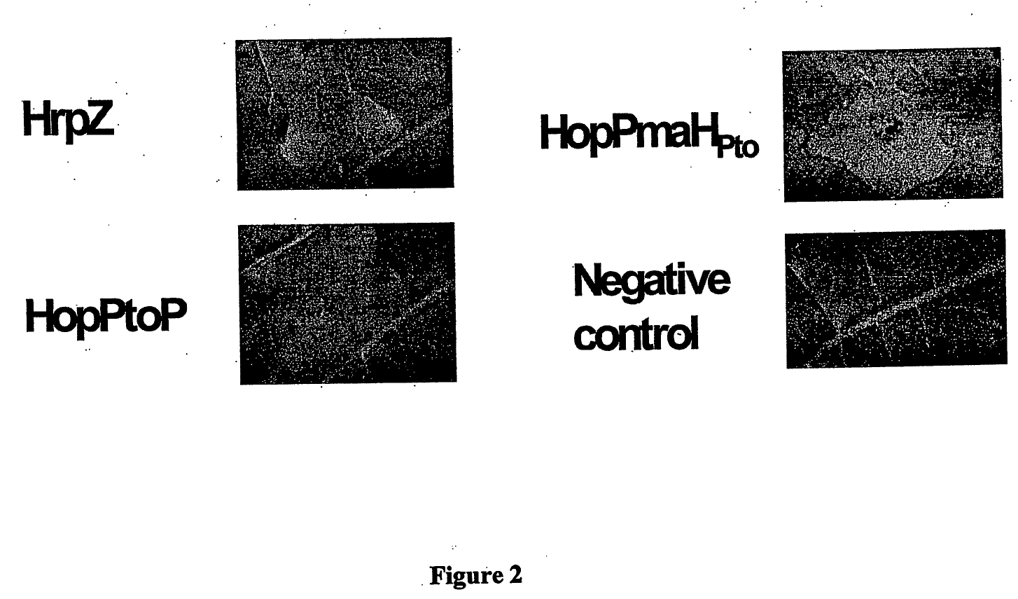

Purified protein preparations prepared in accordance with Example 1 above were used to infiltrate HopPtoP or HopPmaH_{Pto} onto plant tissues. Purified HrpZ was similarly prepared. HrpZ, HopPtoP, and HopPmaH_{Pto} were purified using a 6×His tag system, and denatured at 100° C. for 10 minutes. Proteins were then infiltrated into Nicotiana tabacum cv. xanthi, and the hypersensitive response was photographed at 24 hours (Alfano et al., Mol. Microbiol. 19:715-728 (1996), which is hereby incorporated by reference in its entirety). As shown in FIG. 2, like HrpZ, which is known to elicit a hypersensitive response in tobacco plant tissues, HopPtoP and HopPmaH_{Pto} elicit a similar hypersensitive response, while no such response could be detected from the negative control.

### Example 3

### HopPtoP and HopPmaH_{Pto} Proteins are Secreted in Culture

pENTR/SD/D-TOPO®:: hopPtoP and pENTR/SD/D-TOPO®:: hopPmaH_{Pto} were used to clone into pCPP3234, an IPTG-inducible “Gateway-ized” vector created in the lab by cloning Gateway Reading Frame B fragment into SmaI site of pCPP3214. pCPP3214 has a cyaa construct for gene fusions in broad host range vector pVLT35 (de Lorenzo et al., Gene 123:17-24 (1993), which is hereby incorporated by reference in its entirety) and was constructed by digesting pMJH20 with SacI and HindIII, and ligating this fragment to pVLT35 cut with the same enzymes. This fused the adenylate cyclase (cyaA) gene to the C-terminal ends of hopPtoP and hopPmaH_{Pto}, which provided an eptiope for detection of protein in the cell and supernatant fractions during the secretion assays. The resulting plasmids, pCPP3256 (HopPtoP-CyaA) and pCPP3255 (HopPmaH_{Pto}-CyaA), were then transformed into P. syringae DC3000 and CUCPB5114 (P. syringae DC3000 without the hrp/hrc cluster, negative control).

Bacteria were inoculated in 30 mL Hrp minimal media (induces Hrp system) to OD₆₀₀=0.15 with IPTG to 100 uM. Bacteria were grown under standard conditions and then harvested at OD₆₀₀=0.3. Cultures were centrifuged at 28000 rpm in Beckman L8-70 ultracentrifuge. The top 30 ml supernatant was removed, 7.5 ml trichloroacetic acid (TCA) (˜25% TCA solution) was added to precipitate proteins, and the mixture was incubated at 4° C. overnight (>12 hours). Thereafter, supernatant fractions were centrifuged at 28000 rpm in Beckman L8-70 ultracentrifuge to obtain supernatant protein pellet, which was then washed twice with 5 ml 100% acetone. Protein was resuspended in 1× protein loading buffer, 100 μl for every OD₆₀₀=0.3, and used 20 μl for SDS-PAGE analysis. Also, cell pellets (from above) were resuspended in 1 ml ddH₂O for every OD₆₀₀=0.3, and 15 μl was used for SDS-PAGE analysis. Standard SDS-PAGE analysis was used.

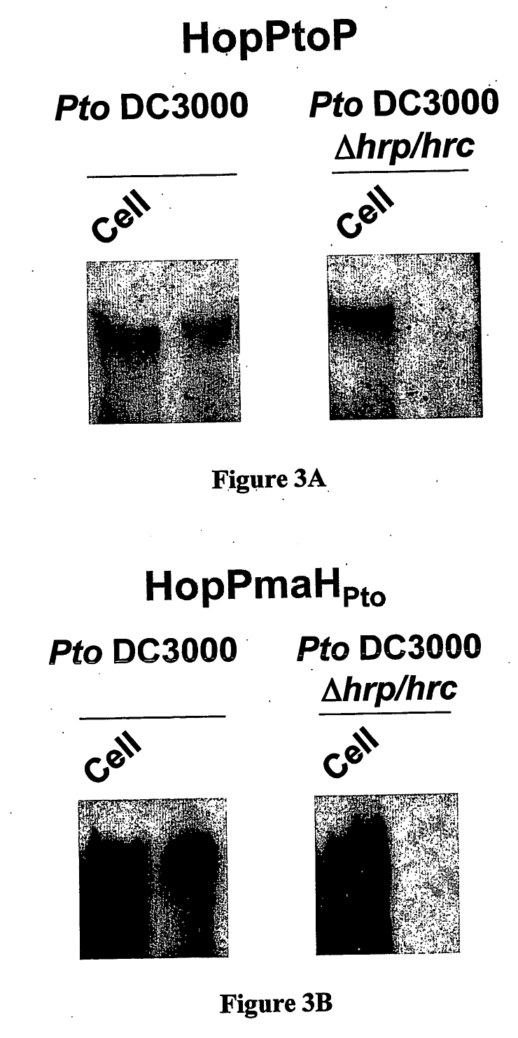

CyaA antibodies (Santa Cruz Biotech; see Lee et al., Infect. Immun. 67(5):2090-2095 (1999), which is hereby incorporated by reference in its entirety) were used to detect HopPtoP and HopPmaH_{Pto} proteins in the cell and supernatant fractions. As shown in FIG. 3A, HopPtoP-CyaA fusion was detected in subsequent Western analysis of the cell pellet (cell) and supernatant (SN) for P. syringae DC3000, which is known to possess a type III secretion system, but only in the cell pellet for P. syringae DC3000 (Δhrp/hrc), which is lacking a type III secretion system. In FIG. 3B, HopPmaH_{Pto}-CyaA fusion was detected in subsequent Western analysis of the cell pellet (cell) and supernatant (SN) for P. syringae DC3000 but only in the cell pellet for P. syringae DC3000 (Δhrp/hrc). Thus, both HopPtoP and HopPmaH_{Pto} fusions were secreted by the type III secretion system of P. syringae of DC3000.

### Example 4

### HopPtoP and HopPmaH_{Pto} are Translocated into Plant Tissues

Vectors pCPP3256 (HopPtoP-CyaA) and pCPP3255 (HopPmaH_{Pto}-CyaA) in P. syringae DC3000, described above, and CUCPB5114 were used for transloction assays. CyaA is the adenylate cyclase domain from the cyclolysin toxin from Bordetella pertussis. This domain uses calmodulin, found only in eukaryotic cells, to produce cAMP from ATP. Therefore, only a CyaA fusion protein that is translocated can produce high levels of cAMP.

Translocation assays were performed by inoculating bacteria in 5 mM MES, pH=5.5 to OD₆₀₀=0.3, to which IPTG was added to 100 μM. The bacteria were infiltrated into tomato cd. Moneymaker. 8 hours post-infiltration, one plant disc was taken from the tomato plant and then crushed with liquid nitrogen. To the crushed plant tissue, 300 μl 0.2M HCl was added and the suspension was frozen at −20° C. The frozen samples were analyzed for cAMP levels with Correlate-EIA Direct cyclic AMP kit from Assay Designs (see Petnicki-Ocwieja et al., Proc. Natl. Acad. Sci. USA 99: 8336-8341 (2002) and accompanying supporting materials available online, which is hereby incorporated by reference in its entirety).

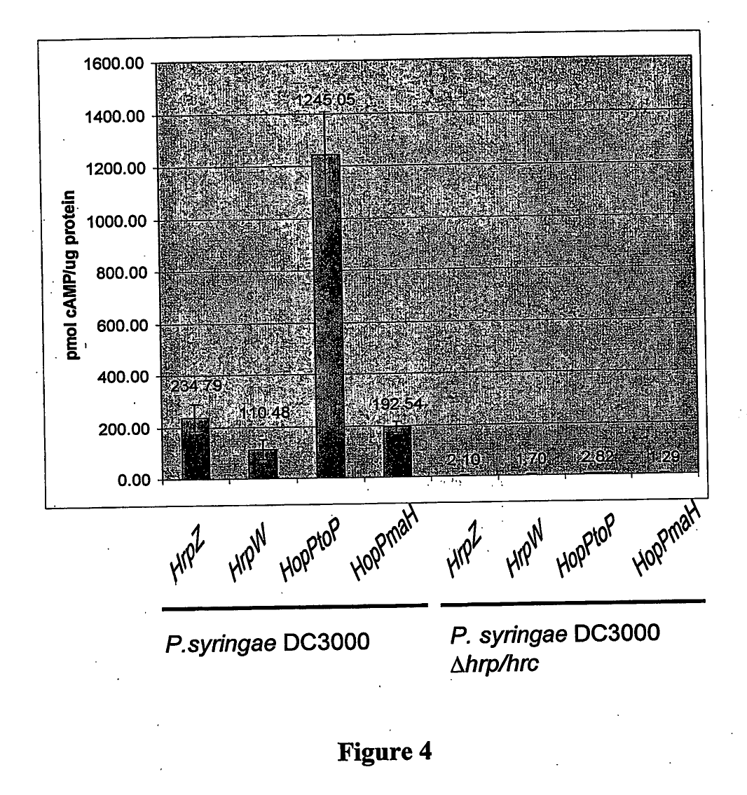

As shown in FIG. 4, HopPtoP and HopPmaH_{Pto}, like the known harpins HrpW and HrpZ, were translocated into tomato leaf tissue, as evidenced by the increased cAMP production by P. syringae DC3000 but not P. syringae DC3000 (Δhrp/hrc). In particular, HopPtoP is translocated at significantly higher levels than either HrpW or HrpZ.

### Example 5

### Transgenic Expression of HopPtoP and HopPmaH_{Pto} in Plants

A variety of technologies have been developed for production of transgenic plants expressing HopPtoP and HopPmaH_{Pto}. Each of these technologies relies on the introduction of one of the recombinant hopPtoP and hopPmaH_{Pto} genes of the present invention into a plant cell or tissue and then regenerating the plant cell or tissue into a transgenic plant. The recombinant hopPtoP and hopPmaH_{Pto} genes can contain either an inducible promoter or a constitutive promoter. Since the introduced gene is limited to a single function, other agronomically important traits of the crop plants remain unmodified. Using this technology, transgenic plants transformed with a recombinant hopPtoP or hopPmaH_{Pto} gene can be propagated and grown under conditions effective to impart disease resistance, enhance plant growth, control insects, and impart stress resistance. Transgenic fruit- or vegetable-bearing plants can be useful for imparting post-harvest disease or desiccation resistance in fruits or vegetables, while transgenic ornamental plants can be useful for imparting desiccation resistance to cuttings of ornamental plants and/or promoting early flowering of ornamental plants.

Although the invention has been described in detail for the purpose of illustration, it is understood that such detail is solely for that purpose, and variations can be made therein by those skilled in the art without departing from the spirit and scope of the invention which is defined by the following claims.

1. An isolated DNA molecule that encodes a hypersensitive response eliciting protein or polypeptide and is selected from the group consisting of
(a) a DNA molecule comprising the nucleotide sequence of SEQ ID NO: 1 or SEQ ID NO: 3,
(b) a DNA molecule encoding a protein or polypeptide comprising the amino acid of SEQ ID NO: 2 or SEQ ID NO: 4, and
(c) a DNA molecule that hybridizes to the complement of SEQ ID NO: 1 or SEQ ID NO: 3 under hybridization conditions including a hybridization medium comprising 5×SSC buffer at a temperature of at least about 42° C. for about 18 hours; or
an isolated DNA molecule that is complementary to at least one of DNA molecules (a), (b), or (c).

2. The isolated DNA molecule according to claim 1, wherein said DNA molecule comprises the nucleotide sequence of SEQ ID NO: 1.

3. The isolated DNA molecule according to claim 1, wherein said DNA molecule encodes a protein or polypeptide comprising the amino acid of SEQ ID NO: 2.

4. The isolated DNA molecule according to claim 1, wherein said DNA molecule hybridizes to the complement of SEQ ID NO: 1 under hybridization conditions including a hybridization medium comprising 5×SSC buffer at a temperature of at least about 42° C. for about 18 hours.

5. The isolated DNA molecule according to claim 1, wherein said DNA molecule comprises the nucleotide sequence of SEQ ID NO: 3.

6. The isolated DNA molecule according to claim 1, wherein said DNA molecule encodes a protein or polypeptide comprising the amino acid of SEQ ID NO: 4.

7. The isolated DNA molecule according to claim 1, wherein said DNA molecule hybridizes to the complement of SEQ ID NO: 3 under hybridization conditions including a hybridization medium comprising 5×SSC buffer at a temperature of at least about 42° C. for about 18 hours.

8. The isolated DNA molecule according to claim 1, wherein said DNA molecule is complementary to at least one of DNA molecules (a), (b), or (c).

9. An expression vector comprising the DNA molecule of claim 1.

10. The expression vector according to claim 9, wherein the DNA molecule is in sense orientation and correct reading frame.

11. A host cell transformed with the DNA molecule of claim 1.

12. The host cell according to claim 11, wherein the host cell is selected from the group consisting of a plant cell and a bacterial cell.

13. The host cell according to claim 11, wherein the DNA molecule is present in an expression vector.

14. A transgenic plant transformed with the DNA molecule of claim 1.

15. The transgenic plant according to claim 14, wherein the plant is selected from the group consisting of alfalfa, rice, wheat, barley, rye, cotton, sunflower, peanut, corn, potato, sweet potato, bean, pea, chicory, lettuce, endive, cabbage, brussel sprouts, beet, parsnip, turnip, cauliflower, broccoli, radish, spinach, onion, garlic, eggplant, pepper, celery, carrot, squash, pumpkin, zucchini, cucumber, apple, pear, melon, citrus, strawberry, grape, raspberry, pineapple, soybean, tobacco, tomato, sorghum, and sugarcane.

16. The transgenic plant according to claim 14, wherein the plant is selected from the group consisting of Arabidopsis thaliana, Saintpaulia, petunia, pelargonium, poinsettia, chrysanthemum, carnation, rose, tulip, and zinnia.

17. A transgenic plant seed transformed with the DNA molecule of claim 1.

18. The transgenic plant seed according to claim 17, wherein the plant seed is selected from the group consisting of alfalfa, rice, wheat, barley, rye, cotton, sunflower, peanut, corn, potato, sweet potato, bean, pea, chicory, lettuce, endive, cabbage, brussel sprouts, beet, parsnip, turnip, cauliflower, broccoli, radish, spinach, onion, garlic, eggplant, pepper, celery, carrot, squash, pumpkin, zucchini, cucumber, apple, pear, melon, citrus, strawberry, grape, raspberry, pineapple, soybean, tobacco, tomato, sorghum, and sugarcane seeds.

19. The transgenic plant seed according to claim 17, wherein the plant seed is selected from the group consisting of Arabidopsis thaliana, Saintpaulia, petunia, pelargonium, poinsettia, chrysanthemum, carnation, rose, tulip, and zinnia seeds.

20. An isolated hypersensitive response eliciting protein or polypeptide selected from the group consisting of a protein or polypeptide having the amino acid comprising SEQ ID NO: 2 or SEQ ID NO: 4, and a protein or polyeptide encoded by a nucleic acid that hybridizes to the complement of SEQ ID NO: 1 or SEQ ID NO: 3 under hybridization conditions including a hybridization medium comprising 5×SSC buffer at a temperature of at least about 42° C. for about 18 hours.

21. The isolated protein or polypeptide according to claim 20, wherein the protein or polypeptide comprises the amino acid sequence of SEQ ID NO: 2.

22. The isolated protein or polypeptide according to claim 20, wherein the protein or polypeptide comprises the amino acid sequence of SEQ ID NO: 4.

23. The isolated protein or polypeptide according to claim 20, wherein the protein or polypeptide is encoded by a nucleic acid that hybridizes to the complement of SEQ ID NO: 1 under hybridization conditions including a hybridization medium comprising 5×SSC buffer at a temperature of at least about 42° C. for about 18 hours.

24. The isolated protein or polypeptide according to claim 20, wherein the protein or polypeptide is encoded by a nucleic acid that hybridizes to the complement of SEQ ID NO: 3 under hybridization conditions including a hybridization medium comprising 5×SSC buffer at a temperature of at least about 42° C. for about 18 hours.

25. A composition comprising:
a protein or polypeptide according to claim 20 and
a carrier.

26. A composition according to claim 25 further comprising an additive selected from the group consisting of fertilizer, insecticide, fungicide, nematacide, and mixtures thereof.

27. A method of imparting disease resistance to plants comprising:
applying a protein or polypeptide according to claim 20 in a non-infectious form to a plant or plant seed under conditions effective to impart disease resistance.

28. The method according to claim 27, wherein said applying is carried out with a plant.

29. The method according to claim 27, wherein said applying is carried out with a plant seed, said method further comprising:
planting the seed treated with the hypersensitive response elicitor in natural or artificial soil and
propagating a plant from the seed planted in the soil.

30. A method of enhancing plant growth comprising:
applying a protein or polypeptide according to claim 20 in a non-infectious form to a plant or plant seed under conditions effective to enhance plant growth.

31. The method according to claim 30, wherein said applying is carried out with a plant.

32. The method according to claim 30, wherein said applying is carried out with a plant seed, said method further comprising:
planting the seed treated with the hypersensitive response elicitor in natural or artificial soil and
propagating a plant from the seed planted in the soil.

33. A method of insect control for plants comprising:
applying a protein or polypeptide according to claim 20 in a non-infectious form to a plant or plant seed under conditions effective to control insects.

34. The method according to claim 33, wherein said applying is carried out with a plant.

35. The method according to claim 33, wherein said applying is carried out with a plant seed, said method further comprising:
planting the seed treated with the hypersensitive response elicitor in natural or artificial soil and
propagating a plant from the seed planted in the soil.

36. A method of imparting post-harvest disease resistance or post-harvest desiccation resistance to a fruit or vegetable comprising:
applying a protein or polypeptide according to claim 20 in a non-infectious form to a fruit or vegetable, a plant, or a plant seed under conditions effective to impart post-harvest disease resistance or desiccation resistance to the fruit or vegetable.

37. The method according to claim 36, wherein said applying is carried out with a fruit or vegetable.

38. The method according to claim 36, wherein said applying is carried out with a plant.

39. The method according to claim 36, wherein said applying is carried out with a plant seed, said method further comprising:
planting the seed treated with the hypersensitive response elicitor in natural or artificial soil and
propagating a plant from the seed planted in the soil.

40. A method of imparting desiccation resistance to cuttings removed from ornamental plants comprising:
applying a protein or polypeptide according to claim 20 in a non-infectious form to an ornamental plant or plant seed, or a cutting removed therefrom, under conditions effective to impart desiccation resistance to cuttings removed from the ornamental plant.

41. The method according to claim 40, wherein said applying is carried out with an ornamental plant.

42. The method according to claim 40, wherein said applying is carried out with a cutting removed from an ornamental plant.

43. The method according to claim 40, wherein said applying is carried out with an ornamental plant seed, said method further comprising:
planting the seed treated with the hypersensitive response elicitor in natural or artificial soil and
propagating a plant from the seed planted in the soil.

44. A method of promoting early flowering of ornamental plants comprising:
applying a protein or polypeptide according to claim 20 in a non-infectious form to an ornamental plant or plant seed under conditions effective to promote flowering of the treated ornamental plants, or plants grown from treated seeds, that occurs earlier as compared to untreated ornamental plants.

45. The method according to claim 44, wherein said applying is carried out with an ornamental plant.

46. The method according to claim 44, wherein said applying is carried out with an ornamental plant seed, said method further comprising:
planting the seed treated with the hypersensitive response elicitor in natural or artificial soil and
propagating a plant from the seed planted in the soil.

47. A method of enhancing the longevity of fruit or vegetable ripeness comprising:
applying a protein or polypeptide according to claim 20 in a non-infectious form to a fruit or vegetable, a plant, or a plant seed under conditions effective to enhance the longevity of fruit or vegetable ripeness.

48. The method according to claim 47, wherein said applying is carried out with a fruit or vegetable.

49. The method according to claim 47, wherein said applying is carried out with a plant.

50. The method according to claim 47, wherein said applying is carried out with a plant seed, said method further comprising:
planting the seed treated with the hypersensitive response elicitor in natural or artificial soil and
propagating a plant from the seed planted in the soil.

51. A method of imparting stress resistance to plants comprising:
applying a protein or polypeptide according to claim 20 in a non-infectious form to a plant or plant seed under conditions effective to impart stress resistance.

52. The method according to claim 51, wherein said applying is carried out with a plant.

53. The method according to claim 51, wherein said applying is carried out with a plant seed, said method further comprising:
planting the seed treated with the hypersensitive response elicitor in natural or artificial soil and
propagating a plant from the seed planted in the soil.

54. A method of imparting disease resistance to plants comprising:
providing a transgenic plant or plant seed transformed with a DNA molecule according to claim 1 and
growing the transgenic plant or transgenic plant produced from the transgenic plant seed under conditions effective to impart disease resistance.

55. The method according to claim 54, wherein a transgenic plant is provided.

56. The method according to claim 54, wherein a transgenic plant seed is provided.

57. A method of enhancing plant growth comprising:
providing a transgenic plant or plant seed transformed with a DNA molecule according to claim 1 and
growing the transgenic plant or transgenic plant produced from the transgenic plant seed under conditions effective to enhance plant growth.

58. The method according to claim 57, wherein a transgenic plant is provided.

59. The method according to claim 57, wherein a transgenic plant seed is provided.

60. A method of insect control for plants comprising:
providing a transgenic plant or plant seed transformed with a DNA molecule according to claim 1 and
growing the transgenic plant or transgenic plant produced from the transgenic plant seed under conditions effective to control insects.

61. The method according to claim 60, wherein a transgenic plant is provided.

62. The method according to claim 60, wherein a transgenic plant seed is provided.

63. A method of imparting post-harvest disease resistance or desiccation resistance to a fruit or vegetable comprising:
providing a transgenic fruit or vegetable plant or plant seed transformed with a DNA molecule according to claim 1 and
growing the transgenic plant or transgenic plant produced from the transgenic plant seed under conditions effective to impart post-harvest disease resistance or desiccation resistance to fruits or vegetables removed from the transgenic plant.

64. The method according to claim 63, wherein a transgenic plant is provided.

65. The method according to claim 63, wherein a transgenic plant seed is provided.

66. A method of enhancing the longevity of fruit or vegetable ripeness comprising:
providing a transgenic fruit or vegetable plant or plant seed transformed with a DNA molecule according to claim 1 and
growing the transgenic plant or transgenic plant produced from the transgenic plant seed under conditions effective to enhance the longevity of ripeness for fruits or vegetables harvested therefrom.

67. The method according to claim 66, wherein a transgenic plant is provided.

68. The method according to claim 66, wherein a transgenic plant seed is provided.

69. A method of imparting desiccation resistance to ornamental plant cuttings comprising:
providing a transgenic ornamental plant or plant seed transformed with a DNA molecule according to claim 1 and
growing the transgenic plant or transgenic plant produced from the transgenic plant seed under conditions effective to impart desiccation resistance to a cutting from the transgenic plant.

70. The method according to claim 69, wherein a transgenic plant is provided.

71. The method according to claim 69, wherein a transgenic plant seed is provided.

72. A method of promoting early flowering of ornamental plants comprising:
providing a transgenic ornamental plant or plant seed transformed with a DNA molecule according to claim 1 and
growing the transgenic plant or transgenic plant produced from the transgenic plant seed under conditions effective to promote early flowering in the transgenic plant.

73. The method according to claim 72, wherein a transgenic plant is provided.

74. The method according to claim 72, wherein a transgenic plant seed is provided.

75. A method of imparting stress resistance to plants comprising:
providing a transgenic plant or plant seed transformed with a DNA molecule according to claim 1 and
growing the transgenic plant or transgenic plant produced from the transgenic plant seed under conditions effective to impart stress resistance to the transgenic plant.

76. The method according to claim 75, wherein a transgenic plant is provided.

77. The method according to claim 75, wherein a transgenic plant seed is provided.

78. A method of imparting desiccation resistance to cuttings removed from ornamental plants comprising:
providing a transgenic ornamental plant or plant seed transformed with a DNA molecule according to claim 1 and
growing the transgenic plant or transgenic plant produced from the transgenic plant seed under conditions effective to impart desiccation resistance to cuttings removed from the ornamental plant.

79. The method according to claim 78, wherein a transgenic plant is provided.

80. The method according to claim 78, wherein a transgenic plant seed is provided.


SEQUENCE LISTING (8 sequences)

SEQ ID NO: 1 - DNA, 975 bp, Pseudomonas syringae
atgaccatgggtgtttcacctattcgtaactcaaactccctgccgatcgatttttcgtcgttgagcgcaaagagtggcgggcataacgggctgggcagcggagacaattcgactatcgacccgagtacgttgttgttcggcaatcaagggcagacgcaggtcaatttcgctccgcccaacagcacggactcctcaaccagcggtgtgaacgctgcgtcaggcaatacggcgtccggcctggtcgagcaaatcatgagcctgctgaaacaattgatgcagatgctgatgcaaaacaacaatgcttccggtaaccctcagactgattcgtcaacgccaggcgtcggcagtggcaacagcgtcgggagcggcggtactggaagcagtctcgcaggcagtgacggtggcgacgaaacgtccggtgtcggtaacggcggtttaggcgacgcgggcagcacgccaacaacgagcgcggccgatggtgtgccctcggatacttcactcacgggtagcggtgggctgcatttgcctcaacagcttgagcagtatcgaggcgacattatggacgccgccaaagccaccggcgtgccgcccagcgtgatcgccgggcagatatgggctgagtcgcgcggtcagttgaatgcggccaccaccaatgtcaacggcaaggccgatgcgggcctgatgcaggtcaacgcagacacgttcaagtcattgcagcagcaaaacccggggttgctgggcaacgacgtcaacgattcgcacaccaacatcatggcgggcgcgctctacctgcgagaccagaacaaggagttcggcgacatgggggcagcacttcgcgcatacaactccgggcccgacaaggtcaataaagccgacctcagcgacacgggaggcgtgggcggcagcagctacccggcggacgtactgaacttcgcgaaaatcatcgagagtgggcagggcaatttacccgcttga

SEQ ID NO: 2 - protein, 324 aa, Pseudomonas syringae
MTMGVSPIRNSNSLPIDFSSLSAKSGGHNGLGSGDNSTIDPSTLLFGNQGQTQVNFAPPNSTDSSTSGVNAASGNTASGLVEQIMSLLKQLMQMLMQNNNASGNPQTDSSTPGVGSGNSVGSGGTGSSLAGSDGGDETSGVGNGGLGDAGSTPTTSAADGVPSDTSLTGSGGLHLPQQLEQYRGDIMDAAKATGVPPSVIAGQIWAESRGQLNAATTNVNGKADAGLMQVNADTFKSLQQQNPGLLGNDVNDSHTNIMAGALYLRDQNKEFGDMGAALRAYNSGPDKVNKADLSDTGGVGGSSYPADVLNFAKIIESGQGNLPA

SEQ ID NO: 3 - DNA, 1668 bp, Pseudomonas syringae
atgaatacgatcaacagaaacatctaccccgtctccgggatttctgcgcaggatgcccctgtacaaactgatcagctccagccgcaaggccagggcatcaggccggggcacaatagcaacctgatcgacttcggactgatacagcaggccaatggtccgcactcatcgctgaacacatcgagctccagaattcagccgactgacaccagcacatcctcaaacaggctggggggtaatggcgatcagttactgaacaaactcgtggaagcgatccgtaatatcctcaacaacctgctctctctgctggaaggcaatcaacaccagggctcttcgcctgcacagacccagcgtgaacagacgccgacgtccactcaatcgcacgcttcgccttcctcgtcgtcttcatcttcgccgtcgacatcctcccagtcttcaccctcagtgccttcaacgcctcagggcaacgcagaaaaaccgtttgtggtgcagagcgatcatccggcggaaaaaccggtatcgctgcagagaacctcagagccaacgtctgtgacgccgccacaaacaccaccgcaggctgtcgagcgaaacagcattaccccggacaaggcaccggccaaacccgaagcggtaaagccggcagtggtcaacgacccggtgctgccgaaaacctcgatccctgccgccgccaagcctgacagcacggtgaccgccgcaaaacacgcgacgcccgctgcccgtggccagggcgctgacatgtccggcatgatcggttttgccaaggaagccaataccaccgggggcaacaacggcgaagtggtcaccgtgaacacggttgccgacctcaagaagtacatggaggacgacaaagcccgcaccgtcaagctgggggccaacctgtctgccgacagtaaagtgtcgataaatttcggggccaacaaaaccctgctgggcaccgataaaggcaacaccctgcacaacatctatctggccagcggcaagaccgccagcaacgacattttccagaatctgaacttcaaccacgacgcccgttaccgtgaaaacggcgacatgcagatgttcatcagcagcggtcagaaatactggatcgaccacatcaccgctaccggaaccaaggatcagaaccccaaaggtctggataaactgctctacgtgggcggcaaggcagataacgtcagcctgaccaattcgaaattccagaacaacgagtatggcgtgattctcggtcagccggacgactcggcagccgccaaagccgagtacaagggctacccacggatgacaatcgccaacaacgtgttcagcaacctcgatgtccgcgggcccggtctgtttcgtcagggccaatttgacgtagttaacaactcgatcgacaaattccacctcggtttcactgcgaccgggaacgctaccatcctgtcgcaggccaactatttcagcaacggtgtcgatgtttccaacaaggcaagtaatagcggcgtgctggatgactacggcgatgcgcacttcaaagacatcggcagtaacgtcagtttcactcagaaatcgccggttaccgcctggacaccgagctacaaccgggacgtgaaaacagccgaagcagccagagcctatgacctggccaatgcgggtgcacaggtcgtgaaataa

SEQ ID NO: 4 - protein, 555 aa, Pseudomonas syringae
MNTINRNIYPVSGISAQDAPVQTDQLQPQGQGIRPGHNSNLIDFGLIQQANGPHSSLNTSSSRIQPTDTSTSSNRLGGNGDQLLNKLVEAIRNILNNLLSLLEGNQHQGSSPAQTQREQTPTSTQSHASPSSSSSSSPSTSSQSSPSVPSTPQGNAEKPFVVQSDHPAEKPVSLQRTSEPTSVTPPQTPPQAVERNSITPDKAPAKPEAVKPAVVNDPVLPKTSIPAAAKPDSTVTAAKHATPAARGQGADMSGMIGFAKEANTTGGNNGEVVTVNTVADLKKYMEDDKARTVKLGANLSADSKVSINFGANKTLLGTDKGNTLHNIYLASGKTASNDIFQNLNFNHDARYRENGDMQMFISSGQKYWIDHITATGTKDQNPKGLDKLLYVGGKADNVSLTNSKFQNNEYGVILGQPDDSAAAKAEYKGYPRMTIANNVFSNLDVRGPGLFRQGQFDVVNNSIDKFHLGFTATGNATILSQANYFSNGVDVSNKASNSGVLDDYGDAHFKDIGSNVSFTQKSPVTAWTPSYNRDVKTAEAARAYDLANAGAQVVK

SEQ ID NO: 5 - DNA, 23 bp, Artificial Sequence
  Description of Artificial Sequence  Primer
caccatgaccatgggtgtttcac

SEQ ID NO: 6 - DNA, 19 bp, Artificial Sequence
  Description of Artificial Sequence  Primer
agcgggtaaattgccctgc

SEQ ID NO: 7 - DNA, 19 bp, Artificial Sequence
  Description of Artificial Sequence  Primer
caccatgaatacgatcaac

SEQ ID NO: 8 - DNA, 15 bp, Artificial Sequence
  Description of Artificial Sequence  Primer
tttcacgacctgtgc